(19) United States
(12) Patent Application Publication
(10) Pub. No.: US 2024/0116900 A1
(43) Pub. Date: Apr. 11, 2024

(54) NEW CRYSTALLINE FORMS OF A KRAS G12C INHIBITOR COMPOUND

(71) Applicant: NOVARTIS AG, Basel (CH)

(72) Inventors: Simona COTESTA, Basel (CH); Heng GE, Changshu, Jiangsu (CN); Marc GERSPACHER, Basel (CH); Catherine LEBLANC, Basel (CH); Bo LIU, Shanghai (CN); Edwige Liliane Jeanne LORTHIOIS, Basel (CH); Rainer MACHAUER, Basel (CH); Robert MAH, Basel (CH); Tanja MEISTER, Basel (CH); Christophe MURA, Basel (CH); Pascal RIGOLLIER, Basel (CH); Nadine SCHNEIDER, Basel (CH); Stefan STUTZ, Basel (CH); Andrea VAUPEL, Basel (CH); Nicolas WARIN, Basel (CH); Rainer WILCKEN, Basel (CH); Lijun XUE, Changshu, Jiangsu (CN); Marie-Anne LOZAC'H, Basel (CH); Ross STRANG, Basel (CH)

(21) Appl. No.: 18/250,466

(22) PCT Filed: Oct. 29, 2021
(86) PCT No.: PCT/CN2021/127601

§ 371 Date: Apr. 25, 2023

(30) Foreign Application Priority Data
Oct. 30, 2020 (WO) PCT/CN2020/125425
Dec. 17, 2020 (WO) PCT/IB2020/062144
Jun. 23, 2021 (WO) PCT/CN2021/101813

(51) Int. Cl. C07D 403/14

(52) U.S. Cl. CPC C07D 403/14

(57) ABSTRACT

Provided are crystalline forms of a KRAS G12C inhibitor compound and to processes for their preparation. Furthermore, provided is pharmaceutical composition comprising said crystalline forms, and at least one pharmaceutically acceptable excipient. The pharmaceutical composition can be used as a medicament, in particular for the treatment of cancer, and KRAS G12C-mutant cancer.

### FIELD OF THE INVENTION

The present invention provides crystalline forms of a therapeutically useful compound, namely 1-{6-[(4M)-4-(5-chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one (Compound A). The present invention also provides a pharmaceutical composition comprising the crystalline forms, as well as methods of preparing and methods of using the crystalline forms in the treatment of cancer and KRAG G12C-mutated cancer, particularly a cancer such as non-small cell lung cancer, colorectal cancer, pancreatic cancer and a solid tumor.

### BACKGROUND

The KRAS oncoprotein is a GTPase with an essential role as regulator of intracellular signaling pathways, such as the MAPK, PI3K and RaI pathways, which are involved in proliferation, cell survival and tumorigenesis. Oncogenic activation of KRAS occurs predominantly through missense mutations in codon 12. KRAS gain-of-function mutations are found in approximately 30% of all human cancers. KRAS G12C mutation is a specific sub-mutation, prevalent in approximately 13% of lung adenocarcinomas, 4% (3-5%) of colon adenocarcinomas and a smaller fraction of other cancer types,

In normal cells, KRAS alternates between inactive GDP-bound and active GTP-bound states. Mutations of KRAS at codon 12, such as G12C, impair GTPase-activating protein (GAP)-stimulated GTP hydrolysis. In that case, the conversion of the GTP to the GDP form of KRAS G12C is therefore very slow. Consequently, KRAS G12C shifts to the active, GTP-bound state, thus driving oncogenic signaling.

A compound which is able to inhibit such oncogenic signaling would therefore be useful. It is also important to be able to provide this compound in the form of a solid form, e.g. a polymorphic form, which is suitable for drug substance and drug product development.

However, it is not yet possible to predict whether a particular compound or salt of a compound will form polymorphs in the first place or whether any such polymorphs will be suitable for commercial use in a pharmaceutical composition which is suitable for administering to patients in need thereof, or which polymorphs will display desirable properties.

This is because different solid state forms of a particular compound often possess different properties. Solid state forms of an active pharmaceutical ingredient (API) thus play an important role in determining the ease of preparation, hygroscopicity, stability, solubility, storage stability, ease of formulation, rate of dissolution in gastrointestinal fluids and in vivo bioavailability of the therapeutic drug.

Processing or handling of the active pharmaceutical ingredient during the manufacture and/or during the formulation process may also be improved when a particular solid form of the API is used. Desirable processing properties mean that certain solid forms can be easier to handle, better suited for storage, and/or allow for better purification.

### SUMMARY

Compound A is the compound of Example 1 and has the chemical structure depicted below,

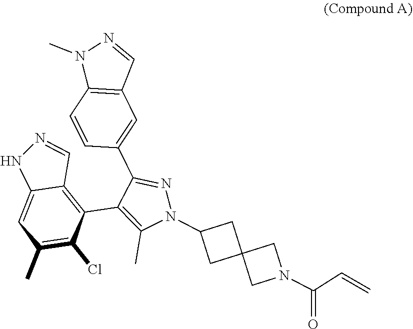

Compound A is a potent and selective covalent inhibitor of KRAS G12C that binds to KRAS G12C and traps it into an inactive guanosine diphosphate (GDP)-bound state. In cellular assays, Compound A selectively inhibited downstream effector protein recruitment to KRAS G12C and inhibited KRAS-driven oncogenic signaling and proliferation specifically in KR ASG12C mutant cell lines. In KRAS G12C mutant xenograft and patient-derived xenograft tumor models in mice, Compound A treatment also resulted in dose-dependent antitumor activity, KRAS G12C target occupancy, and reduction of expression of the mitogen-activated protein kinase (MAPK) pathway target gene, dual-specific phosphatase 6 (DUPS6).

Compound A therefore has the potential to reduce tumor growth in patients with KRAS G12C mutant solid tumors.

Obtaining crystalline forms of Compound A has not been straightforward. Routine crystallization experiments with Compound A such as evaporation from hot saturated solutions or by precipitation only gave amorphous material, oils or gel-like material.

The present inventors have now been able to produce crystalline solid forms of Compound A which have properties which render them suitable for use in drug substance and drug product development. These solid forms provide handling properties which are suitable for manufacture on an industrial scale. The present invention also provides methods of producing these polymorphs which are amenable to large-scale production.

The forms provided herein have good physical and chemical stability and/or have good processing qualities. In addition, some of the forms provided herein are useful as intermediates which enable other useful crystalline forms of Compound A to be made.

The present invention provides a crystalline form of the Compound A, as defined herein, which is selected from Hydrate HA crystalline form, Hydrate HB crystalline form, Hydrate HC crystalline form, Modification C crystalline form, a lactic acid solvate form (e.g. Form G of the L-lactic acid solvate crystalline form or Form F of L-lactic acid solvate crystalline) and an alcohol solvate (e.g. an isopropyl alcohol solvate, an ethanol solvate, a methanol solvate, a propylene glycol solvate, a 1-butanol solvate or an n-propanol solvate) crystalline form of Compound A.

The present invention provides a crystalline form of the Compound A, as defined herein, which is selected from Hydrate HA crystalline form, Hydrate HB crystalline form, Hydrate HC crystalline form, Modification C crystalline form, Form G of the L-lactic acid solvate crystalline form Form F of L-lactic acid solvate crystalline and an alcohol solvate (e.g. an isopropyl alcohol solvate, an ethanol solvate, a methanol solvate, a propylene glycol solvate, a 1-butanol solvate or an n-propanol solvate) crystalline form of Compound A.

The present invention also provides a crystalline form of Compound A, as defined herein, having an X-ray powder diffraction spectrum substantially the same as the X-ray powder diffraction spectrum shown in FIG. 1, FIG. 2, FIG. 3, FIG. 4, FIG. 5, FIG. 6, FIG. 7, FIG. 8, FIG. 9, FIG. 10, FIG. 11, or FIG. 12.

The present invention also provides a crystalline form of Compound A, as defined herein, having an X-ray powder diffraction spectrum substantially the same as the X-ray powder diffraction spectrum shown in FIG. 1, FIG. 2, FIG. 3, FIG. 4, FIG. 5, FIG. 6, FIG. 7, FIG. 8, FIG. 9, FIG. 10, FIG. 11, or FIG. 12, when measured using CuKα radiation.

### BRIEF DESCRIPTION OF THE FIGURES

FIG. 1. illustrates the x-ray powder diffraction pattern of Hydrate HA of a (R)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one (Compound A).

FIG. 2. illustrates the x-ray powder diffraction pattern of the isopropyl alcohol (IPA) solvate of a (R)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one (Compound A).

FIG. 3. illustrates the x-ray powder diffraction pattern of the ethanol (EtOH) solvate of a (R)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one (Compound A).

FIG. 4. illustrates the x-ray powder diffraction pattern of the methanol solvate of a (R)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one (Compound A) which has partially converted to Hydrate HA.

FIG. 5. illustrates the x-ray powder diffraction pattern of the propylene glycol solvate of a (R)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one (Compound A).

FIG. 6. illustrates the x-ray powder diffraction pattern of the 1-butanol solvate of a (R)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one (Compound A).

FIG. 7. illustrates the x-ray powder diffraction pattern of the n-propanol solvate of a (R)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one (Compound A).

FIG. 8. illustrates the x-ray powder diffraction pattern of Modification C of a (R)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one (Compound A).

FIG. 9. illustrates the x-ray powder diffraction pattern of Hydrate HB of a (R)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one (Compound A).

FIG. 10. illustrates the x-ray powder diffraction pattern of Hydrate HC of a (R)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one (Compound A).

FIG. 11. illustrates the x-ray powder diffraction pattern of Form G of the L-lactic acid solvate of a (R)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one (Compound A).

FIG. 12. illustrates the x-ray powder diffraction pattern of Form F of the L-lactic acid solvate of a (R)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one (Compound A).

FIG. 13 illustrates the water sorption-desorption isotherm of Modification C of Compound A.

FIG. 14 illustrates the water sorption-desorption isotherm of Hydrate HA of Compound A.

FIG. 15 illustrates the water sorption-desorption isotherm of Hydrate HB of Compound A.

### DETAILED DESCRIPTION OF THE INVENTION

The invention provides crystalline forms of Compound A which are described and characterized herein.

The chemical name of Compound A is 1-{6-[(4M)-4-(5-chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one.

Compound A is the compound with the following chemical structure. Compound A is also known by the name “a (R)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one”.

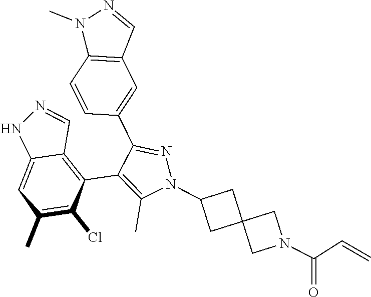

Compound A is also known as “JDQ443” or “NVP-JDQ443” and is described in Example 1 of PCT application WO2021/124222, published 24 Jun. 2021.

For manufacturing pharmaceutical compounds and their formulations, it is important that the active compound is in a form that can be conveniently handled and processed in order to obtain a commercially viable, reliable, and reproducible manufacturing process.

It has been found that certain crystalline forms of Compound A, in particular Hydrate HA and Modification C of the present invention, possess favorable physicochemical properties which are particularly useful for a drug substance intended for use in an oral solid dosage form.

Various embodiments or aspects of the invention are described herein and in particular in the claims. It will be recognized that features specified in each embodiment may be combined with other specified features to provide further embodiments of the present invention. In particular, it will be recognized that features referred to in a particular embodiment or aspect are preferred aspects of the invention. The following embodiments are representative of the invention.

Any one of the crystalline forms of the present invention may be characterized by an X-ray powder diffraction pattern with one, two, three, four, five, six, seven, eight, or more, or all of the peaks in the Table associated with that crystalline form in the Examples below. For example, each form may be characterized by an X-ray powder diffraction pattern with at least one, two, three or four peaks, (for example four) especially peaks chosen from the most characteristic peaks.

The crystalline forms of the present invention may be characterized by analytical methods well known in the field of the pharmaceutical industry for characterizing solids. Such methods comprise but are not limited to melting point determination, PXRD, DSC and TGA.

A given crystalline form may be characterized by one of the aforementioned analytical methods or by combining two or more of them. In particular, Hydrate HA and/or Modification C of Compound A may be characterized by any one of the features or by combining two or more of the features described herein.

### Hydrate HA of Compound A

Hydrate HA of Compound A is a solid form with advantageous properties and is suitable for processing into a drug product which can be administered to a subject in need thereof.

Hydrate HA of Compound A is also referred to herein as “crystalline form Hydrate HA of Compound A”.

Hydrate HA remains unchanged to a large extent upon variation of humidity and temperature. For example, the XRPD pattern of Hydrate HA remains unchanged when heated at ambient relative humidity from 25° C. to 65° C. At 80° C., it converts into an anhydrous form, namely Modification A. Modification A converts back to Hydrate HA when the relative humidity is increased to 10% relative humidity (RH) or above.

In contrast, the XRPD pattern of Hydrate HB changes to hydrate HC when heated to a lower temperature, i.e. from 25° C. to 40° C., and converts to an anhydrous form, Modification B, upon further heating to 70° C. and above.

The increase in stability of Hydrate HA in the presence of moisture and temperature makes Hydrate HA more attractive than other forms, (for example, Hydrate HB), for the development of a solid dosage form with crystalline drug substance.

Hydrate HA of Compound A may be characterized by an x-ray powder diffraction pattern (XRPD) comprising at least one, two, three or four peaks having an angle of refraction 2θ values (CuKα λ=1.5418 Å) selected from the group consisting of 8.2°, 11.6°, 12.9° and 18.8°, measured at a temperature of about 25° C. and an x-ray wavelength, λ, of 1.5418 Å. Hydrate HA of Compound A may be characterized by an x-ray powder diffraction pattern (XRPD) comprising peaks having an angle of refraction 2θ values (CuKα λ=1.5418 Å) selected from the group consisting of 8.2°, 11.6°, 12.9° and 18.8°, measured at a temperature of about 25° C. and an x-ray wavelength, 1, of 1.5418 Å.

Hydrate HA crystalline form may also be characterized by an x-ray powder diffraction pattern (XRPD) comprising at least one, two, three, four, five, six, seven, or eight, or all peaks having an angle of refraction 2θ values (CuKα λ=1.5418 Å) selected from the group consisting of 8.2°, 11.6°, 12.1°, 12.9°, 14.6°, 16.2°, 18.8θ, 20.4° and 24.1°, measured at a temperature of about 25° C. and an x-ray wavelength, 1, of 1.5418 Å. Hydrate HA crystalline form may also be characterized by an x-ray powder diffraction pattern (XRPD) comprising at least four or five peaks having an angle of refraction 2θ values (CuKα λ=1.5418 Å) selected from the group consisting of 8.2°, 11.6°, 12.1°, 12.9°, 14.6°, 16.2°, 18.8°, 20.4° and 24.1°, measured at a temperature of about 25° C. and an x-ray wavelength, λ, of 1.5418 Å.

In one embodiment, Hydrate HA is present in substantially pure form.

The differential scanning calorimetry (DSC) of Hydrate HA shows two endothermic events with peak temperatures at around 28° C. and 78° C., when heated at 10 K/min. The thermal events are most likely associated to dehydration and melting. Upon further heating the sample shows a glass transition at about 138° C.

Hydrate HA is hygroscopic and absorbs up to 7.0% at 80% RH at 25° C. Hydrate HA was stable after equilibration in most solvents at 25° C., with no form change observed. Granulation simulation experiments carried out with water as the solvent for granulation showed that there was no form change of Hydrate HA, unlike Hydrate HB.

The present invention also provides a process for the manufacture of Hydrate HA which can be carried out on an industrial scale. Hydrate HA may be manufactured by first forming an alcoholic solvate, (e.g., an isopropyl alcohol solvate, an ethanol solvate, a methanol solvate, a propylene glycol solvate, a 1-butanol solvate or an n-propanol solvate) of Compound A and leaving the alcoholic solvate to convert spontaneously into Hydrate HA upon exposure to air. Alternatively, Hydrate HA may be manufactured by first forming the ethanolic solvate from another alcoholic solvate e.g., an isopropyl alcohol solvate, a methanol solvate, a propylene glycol solvate, a 1-butanol solvate or an n-propanol solvate) of Compound A and leaving the ethanolic solvate to convert spontaneously into Hydrate HA upon exposure to air.

The present invention thus provides the use of an alcoholic solvate (e.g., an isopropyl alcohol solvate, an ethanol solvate, a methanol solvate, a propylene glycol solvate, a 1-butanol solvate or an n-propanol solvate) of Compound A in the manufacture of Hydrate HA.

The present invention provides a process for the preparation of crystalline form Hydrate HA of Compound A comprising the steps:
(i) suspending Compound A in an alcoholic solvent to form the corresponding alcoholic solvate;
(ii) separating at least a part of the crystals obtained from the mother liquor;
(iii) optionally washing the isolated crystals; and
(iv) drying the isolated crystals under reduced pressure in a humid atmosphere to form Hydrate HA crystalline form.

There is also provided a process for the preparation of crystalline form Hydrate HA of Compound A comprising the steps:
(i) suspending Compound A in an alcohol to form the corresponding alcoholic solvate of Compound A in crystalline form;
(ii) separating at least a part of the crystals obtained from the mother liquor;
(iii) optionally washing the isolated crystals; and
(iv) drying the separated crystals (optionally drying under reduced pressure) in a humid atmosphere to form Hydrate HA crystalline form.

The present invention provides a process for the manufacture of Hydrate HA comprising the steps of: (i) dissolving Compound A in an alcoholic solvent mixture (e.g. a mixture of tetrahydrofuran and ethanol); (ii) forming a concentrated solution of Compound A in the solvent mixture by removing some of the solvent mixture; (iii) adding alcoholic solvate crystals or Hydrate HA crystals as seed crystals to the resulting solution; (iv) heating the resulting mixture (e.g. to a temperature between 40 to 70° C.); (v) removing the remaining solvent to form a wet cake of the alcoholic solvate of Compound A (e.g. the ethanolic alcoholic solvate of Compound A when a mixture of tetrahydrofuran and ethanol is used) and (v) drying the wet cake at a temperature ranging from room temperature to 60° C. (e.g. 50° C.), Linder controlled vacuum (e.g. 30 to 60 mbar) under a water vapor atmosphere.

There is provided a process for the manufacture of Hydrate HA comprising the steps of (i) dissolving Compound A in an ethanol solvent mixture comprising ethanol and a solvent with a lower boiling point than ethanol (e.g., dichloromethane or tetrahydrofuran); (ii) removing the solvent with the lower boiling point (e.g. dichloromethane or tetrahydrofuran) to form a concentrated solution of Compound A in ethanol; (iii) adding more ethanol to the mixture; (iv) adding ethanol solvate crystals as seed crystals to the resulting solution; (iv) heating the resulting mixture (e.g. to a temperature between 40 to 70° C.); (v) removing the ethanol to form a wet cake of ethanol solvate of Compound A and (v) drying the wet cake at a temperature ranging from room temperature to 60° C. (e.g. 50° C.), under controlled vacuum (e.g. 30 to 60 mbar) under a water vapor atmosphere.

### Alcoholic Solvates of Compound A

Hydrate HA of Compound A is obtained via a solid-solid transition via an alcoholic solvate of Compound A. For example, Hydrate HA of Compound A can be obtained from an isopropyl alcohol solvate, an ethanol solvate, a methanol solvate, a propylene glycol solvate, a 1-butanol solvate or an n-propanol solvate of Compound A by exposure to air. An alcoholic solvate of Compound A may therefore be particularly useful as a starting material for the manufacture of Hydrate HA.

In one embodiment, the alcoholic solvate is present in substantially pure form.

The isopropyl alcohol (IPA) solvate of Compound A may be characterized by an x-ray powder diffraction pattern (XRPD) comprising at least two or three peaks having an angle of refraction 2θ values (CuKαλ=1.5418 Å) selected from the group consisting of 7.5°, 12.5° and 17.6° measured at a temperature of about 25° C. and an x-ray wavelength, λ, of 1.5418 Å. The isopropyl alcohol solvate of Compound A may also be characterized by an x-ray powder diffraction pattern (XRPD) comprising at least one, two, three, four, five or six, or all peaks having an angle of refraction 2θ values (CuKα λ=1.5418 Å) selected from the group consisting of 7.5°, 12.5°, 15.5°, 16.4°, 17.6°, 21.4° and 24.4°, measured at a temperature of about 25° C. and an x-ray wavelength, λ, of 1.5418 Å.

The ethanol (EtOH) solvate of Compound A may be characterized by an x-ray powder diffraction pattern (XRPD) comprising at least two, or three or four peaks having an angle of refraction 2θ values (CuKα λ=1.5418 Å) selected from the group consisting of 7.9, 12.7°, 18.2° and 23.1°, measured at a temperature of about 25° C. and an x-ray wavelength, λ, of 1.5418 Å. The ethanol solvate of Compound A may be characterized by an x-ray powder diffraction pattern (XRPD) comprising at least one, two, three, four, five, six, seven, or eight, or more, or all peaks having an angle of refraction 2θ values (CuKα λ=1.5418 Å) selected from the group consisting of 7.9°, 12.70, 13.10, 15.5°, 15.9, 16.9°, 18.2°, 18.6°, and 23.1°, measured at a temperature of about 25° C. and an x-ray wavelength, λ, of 1.5418 Å.

The propylene glycol solvate of Compound A may be characterized by an x-ray powder diffraction pattern (XRPD) comprising at least two, or three or four peaks having an angle of refraction 2θ values (CuKα λ=1.5418 Å) selected from the group consisting of 7.3°, 13.2°, 18.0° and 22.5°, measured at a temperature of about 25° C. and an x-ray wavelength, λ, of 1.5418 Å. The propylene glycol solvate of Compound A may also be characterized by an x-ray powder diffraction pattern (XRPD) comprising at least one, two, three, four, five, six, seven, or eight, or more, or all peaks having an angle of refraction 2θ values (CuKα λ=1.5418 Å) selected from the group consisting of 7.3°, 13.2°, 15.6°, 16.2°, 18.0°, 22.5°, 22.8°, 23.2° and 25.1°, measured at a temperature of about 25° C. and an x-ray wavelength, λ, of 1.5418 Å. The propylene glycol solvate of Compound A may also be characterized by an x-ray powder diffraction pattern (XRPD) comprising at least one, two, three, four, five, six, seven, or eight, or more, or all peaks having an angle of refraction 2θ values (CuKα λ=1.5418 Å) selected from the corresponding Table in Example 2e, measured at a temperature of about 25° C. and an x-ray wavelength, λ, of 1.5418 Å.

The 1-butanol solvate of Compound A may be characterized by an x-ray powder diffraction pattern (XRPD) comprising at least two, or three or four peaks having an angle of refraction 20 values (CuKα λ=1.5418 Å) selected from the group consisting of 7.7°, 14.5°, 17.9° and 19.3°, measured at a temperature of about 25° C. and an x-ray wavelength, λ, of 1.5418 Å. The 1-butanol solvate of Compound A may also be characterized by an x-ray powder diffraction pattern (XRPD) comprising at least one, two, three, four, five, six, seven, or eight, nine, or more, or all peaks having an angle of refraction 2θ values (CuKα λ=1.5418 Å) selected from the group consisting of 7.7°, 12.80, 14.5°, 15.7°, 17.9°, 19.3°, 21.3°, 22.2°, 24.00 and 28.8°, measured at a temperature of about 25° C. and an x-ray wavelength, λ, of 1.5418 Å.

The n-propanol solvate of Compound A may be characterized by an x-ray powder diffraction pattern (XRPD) comprising at least two, or three or four peaks having an angle of refraction 20 values (CuKα λ=1.5418 Å) selected from the group consisting of 7.6°, 15.3°, 17.7° and 18.5°, measured at a temperature of about 25° C. and an x-ray wavelength, λ, of 1.5418 Å. The n-propanol solvate of Compound A may also be characterized by an x-ray powder diffraction pattern (XRPD) comprising at least one, two, three, four, five, six, seven, or eight, or more, or all peaks having an angle of refraction 2θ values (CuKα λ=1.5418 Å) selected from the group consisting of 7.6°, 12.3°, 13.1°, 15.3°, 16.0°, 16.7°, 17.7°, 18.5° and 28.1°, measured at a temperature of about 25° C. and an x-ray wavelength, λ, of 1.5418 Å.

### Hydrate HB of Compound A

Hydrate HB of Compound A may be obtained directly by crystallization from a mixture of methanol/water (60:40), instead of requiring formation of an alcoholic solvate at first.

Hydrate HB is a tetrahydrate (theoretical water content of 12.1%) and is also referred to as “tetrahydrate HB”.

Hydrate HB converts readily into another hydrate, Hydrate HC (which is also referred to as monohydrate HC). Below 30% relative humidity (RH), Hydrate HB converts into a monohydrate HC and completely dehydrates at 0% RH into an anhydrous form which forms Hydrate HC when the relative humidity is raised to 20% RH or above. Monohydrate HC converts to the tetrahydrate HB when the relative humidity is increased to above 60%-70% RH.

Hydrate HB of Compound A may be characterized by an x-ray powder diffraction pattern (XRPD) comprising at least two, three or all peaks having an angle of refraction 2θ values (CuKα λ=1.5418 Å) selected from the group consisting of 7.9°, 15.8°, 18.2°, and 26.4°. measured at a temperature of about 25° C. and an x-ray wavelength, λ, of 1.5418 Å. Hydrate HB of Compound A may also be characterized by an x-ray powder diffraction pattern (XRPD) comprising at least one, two, three, four, five, six, seven, eight, or all peaks having an angle of refraction 2θ values (CuKα λ=1.5418 Å) selected from the group consisting of 6.5°, 7.9°, 12.0°, 13.1°, 15.8°, 17.2°, 17.7°, 18.2°, 19.8°, 21.6°, 23.1° and 26.4° measured at a temperature of about 25° C. and an x-ray wavelength, λ, of 1.5418 Å.

### Modification C of Compound A

Modification C of Compound A is a stable anhydrous crystalline form with a melting point at about 196° C. when heated in a DSC at 10 K/min in a sample pan with a pin hole. Melting is associated with decomposition. Modification C is non hygroscopic and shows a maximum water uptake of 0.5% at 95% RH. Modification C can be obtained by crystallization from ethyl acetate/heptane, but requires highly pure starting material for crystallization. Modification C shows needle shaped particle morphology. Modification C was stable after equilibration in most solvents at 25° C., 50° C. or 70° C., except in ethanol, and methanol where it converts to Hydrate HA, and in isopropanol where it converts into a mixture of HA and the isopropanol solvate.

Granulation simulation experiments carried out with aqueous media as the solvent for granulation showed that there was no form change of Modification C. This was not the case for Hydrate HB of Compound A.

In one embodiment, Modification C is present in substantially pure form.

Modification C of Compound A may be characterized by an x-ray powder diffraction pattern (XRPD) comprising at least one, two, three or all peaks having an angle of refraction 2θ values (CuKα λ=1.5418 Å) selected from the group consisting of 6.1°, 12.2°, 16.3°, and 19.4° measured at a temperature of about 25° C. and an x-ray wavelength, λ, of 1.5418 Å.

Modification C of Compound A may be characterized by an x-ray powder diffraction pattern (XRPD) comprising peaks having an angle of refraction 2θ values (CuKα λ=1.5418 Å) selected from the group consisting of 6.1°, 12.2°, 16.3°, and 19.4° measured at a temperature of about 25° C. and an x-ray wavelength, λ, of 1.5418 Å.

Modification C of Compound A may also be characterized by an x-ray powder diffraction pattern (XRPD) comprising at least one, two, three, four, five, six, seven, eight, or more, or all peaks having an angle of refraction 2θ values (CuKα λ=1.5418 Å) selected from the group consisting of 6.1°, 7.3°, 8.8°, 12.2°, 14.7°, 15.4°, 16.3°, 18.2°, 19.4°, 20.8°, 21.8°, 25.4° and 29.4° measured at a temperature of about 25° C. and an x-ray wavelength, λ, of 1.5418 Å.

### L-Lactic Acid Solvate Forms of Compound A

L-lactic acid solvate forms F and G of Compound A as described herein are also physically stable crystalline forms of Compound A and may thus be incorporated into pharmaceutical compositions comprising Compound A.

### Definitions

In the context of the present invention the following definitions have the indicated meaning, unless explicitly stated otherwise.

As used herein, the term “crystalline form” of Compound A refer to a crystalline solvate, or a crystalline hydrate of Compound A. The term “crystalline forms” is to be construed accordingly.

As used herein the term “polymorph” refers to crystalline forms having the same chemical composition but different spatial arrangements of the molecules, atoms, and/or ions forming the crystal.

The terms “anhydrous form” or “anhydrate” as used herein refer to a crystalline solid where no water is cooperated in or accommodated by the crystal structure. Anhydrous forms may still contain residual water, which is not part of the crystal structure but may be adsorbed on the surface or absorbed in disordered regions of the crystal. Typically, an anhydrous form does not contain more than 2.0 w %, preferably not more than 1.0 w % of water, based on the weight of the crystalline form.

The term “hydrate” as used herein, refers to a crystalline solid where either water is cooperated in or accommodated by the crystal structure e.g., is part of the crystal structure or entrapped into the crystal (water inclusions). Thereby, water can be present in a stoichiometric or non-stoichiometric amount. For example, a hydrate may be referred to as a hemihydrate or as a monohydrate depending on the water/compound stoichiometry. The water content can be measured, for example, by Karl-Fischer-Coulometry.

As used herein, the term “amorphous” refers to a solid form of a compound that is not crystalline. An amorphous compound possesses no long-range order and does not display a definitive X-ray diffraction pattern with reflections.

As used herein, the term “room temperature” refers to a temperature in the range of from 20 to 30° C.

Measurements are taken under standard conditions common in the art, unless specified otherwise.

The term “substantially the same” with reference to X-ray diffraction peak positions means that typical peak position and intensity variability are taken into account. For example, one skilled in the art will appreciate that the peak positions (two-theta (2θ values) will show some inter-apparatus variability, typically as much as 0.2° or 0.1°.

It will be understood that two-theta (2θ) values quoted herein may be plus or minus 0.2° 2θ of the numerical values quoted. Further, one skilled in the art will appreciate that relative peak intensities will show inter-apparatus variability as well as variability due to degree of crystallinity, preferred orientation, prepared sample surface, and other factors known to those skilled in the art and that relative peak intensities should be taken as qualitative measures only.

Unless stated otherwise, two-theta (2θ values) quoted herein are measured at a temperature of about 25° C. and an x-ray wavelength, λ, of 1.5418 Å.

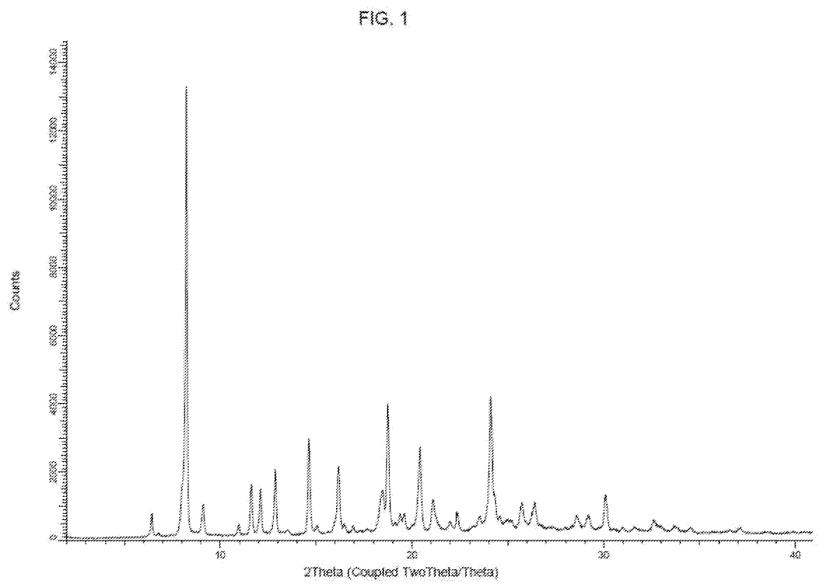

An expression referring to Hydrate HA having “an X-ray powder diffraction pattern substantially the same as the X-ray powder diffraction pattern shown in FIG. 1” may be interchanged with an expression referring to a crystalline Hydrate HA having “an X-ray powder diffraction pattern characterised by the representative X-ray powder diffraction pattern shown in FIG. 1”. Similar expressions referring to other forms of Compound A as described herein should be construed accordingly.

One of ordinary skill in the art will also appreciate that an X-ray diffraction pattern may be obtained with a measurement error that is dependent upon the measurement conditions employed.

In particular, it is generally known that intensities in an X-ray diffraction pattern may fluctuate depending upon measurement conditions employed. It should be further understood that relative intensities may also vary depending upon experimental conditions and, accordingly, the exact order of intensity should not be taken into account. Additionally, a measurement error of diffraction angle for a conventional X-ray diffraction pattern is typically about 5% or less, and such degree of measurement error should be taken into account as pertaining to the aforementioned diffraction angles. Consequently, it is to be understood that the crystal form of the instant invention is not limited to the crystal form that provides an X-ray diffraction pattern completely identical to the X-ray diffraction pattern depicted in the accompanying Figures disclosed herein. Any crystal forms that provide X-ray diffraction patterns substantially identical to that disclosed in the accompanying Figures fall within the scope of the present invention. The ability to ascertain substantial identities of X-ray diffraction patterns is within the purview of one of ordinary skill in the art.

The crystalline forms or solvates of Compound A may be referred to herein as being characterized by graphical data “as shown in” a figure. Such data include, for example, powder X-ray diffraction, DSC and TGA analysis. The person skilled in the art understands that factors such as variations in instrument type, response and variations in sample directionality, sample concentration and sample purity may lead to small variations for such data when presented in graphical form, for example variations relating to the exact peak positions and intensities. However, a comparison of the graphical data in the figures herein with the graphical data generated for another or an unknown solid form and the confirmation that two sets of graphical data relate to the same crystal form is well within the knowledge of a person skilled in the art.

As used herein, the term “mother liquor” refers to the solution remaining after crystallization of a solid from said solution.

As used herein, “substantially pure” or “essentially pure form” when used in reference to a form disclosed herein, e.g., Hydrate HA or Modification C, means the compound having a purity greater than 90 weight % (w %), including greater than 90, 91, 92, 93, 94, 95, 96, 97, 98, and 99 w %, and also including equal to about 100 w % of Compound A, based on the weight of the compound. The remaining material comprises other form(s) of the compound, and/or reaction impurities and/or processing impurities arising from its preparation. For example, a crystalline form of Compound A may be deemed substantially pure in that it has a purity greater than 90 w %, as measured by means that are at this time known and generally accepted in the art, where the remaining less than 10 w % of material comprises other form(s) of Compound A and/or reaction impurities and/or processing impurities. Thus, in an embodiment, provided is a crystalline form of Compound A, (e.g. Hydrate HA, or Modification C) having a purity greater than 90 w %, including greater than 90, 91, 92, 93, 94, 95, 96, 97, 98, and 99 w %.

The term “pharmaceutically acceptable excipient” as used herein refers to substances, which do not show a significant pharmacological activity at the given dose and that are added to a pharmaceutical composition in addition to the active pharmaceutical ingredient. Excipients may take the function of vehicle, diluent, release agent, disintegrating agent, dissolution modifying agent, absorption enhancer, stabilizer or a manufacturing aid among others. Excipients may include fillers (diluents), binders, disintegrants, lubricants and glidants.

The terms “filler” or “diluent” as used herein refer to substances that are used to dilute the active pharmaceutical ingredient prior to delivery. Diluents and fillers can also serve as stabilizers.

As used herein the term “binder” refers to substances, which bind the active pharmaceutical ingredient and pharmaceutically acceptable excipient together to maintain cohesive and discrete portions.

The terms “disintegrant” or “disintegrating agent” as used herein refers to substances, which, upon addition to a solid pharmaceutical composition, facilitate its break-up or disintegration after administration and permits the release of the active pharmaceutical ingredient as efficiently as possible to allow for its rapid dissolution.

The term “lubricant” as used herein refers to substances, which are added to a powder blend to prevent the compacted powder mass from sticking to the equipment during tableting or encapsulation process. They help the ejection of the tablet from the dies and can improve powder flow.

The term “glidant” as used herein refers to substances, which are used for tablet and capsule formulations to improve flow properties during tablet compression and to produce an anti-caking effect.

The term “effective amount” or “therapeutically effective amount” as used herein with regard to Compound A, which causes the desired therapeutic and/or prophylactic effect.

The term “non-hygroscopic” as used herein refers to a compound showing a water uptake of at most 2 w % in the sorption cycle when measured with GMS (or DVS) at a relative humidity in the range of from 0 to 95% RH and a temperature of (25.0±0.1) ° C., based on the weight of the compound. Non-hygroscopic is preferably up to 0.5%.

The terms “solid form” or “solid state form” as used herein interchangeably refer to any crystalline and/or amorphous phase of a compound.

Pharmaceutical Compositions and Uses

In a further aspect the present invention provides the use of a crystalline form of Compound A as defined in any one of the aspects and their corresponding embodiments described above for the preparation of a pharmaceutical composition.

In yet another aspect, the present invention provides a pharmaceutical composition comprising a crystalline form of Compound A as defined in any one of the aspects and their corresponding embodiments described above, and optionally at least one pharmaceutically acceptable excipient.

The at least one pharmaceutically acceptable excipient, which is comprised in the pharmaceutical composition of the present invention, is preferably selected from the group consisting of fillers, diluents, binders, disintegrants, lubricants, glidants and combinations thereof.

In a preferred embodiment, the pharmaceutical composition comprising a crystalline form of Compound A as defined in any one of the aspects and their corresponding embodiments described above is an oral solid dosage form such as a tablet.

In a further aspect, the present invention provides the crystalline form of Compound A or the pharmaceutical composition comprising the same as defined in any one of the described aspects described herein and their corresponding embodiments for use as a medicament.

In yet another aspect, the present invention provides a crystalline form of Compound A, or pharmaceutical composition comprising the same as defined in any one of the aspects described herein and their corresponding embodiments for use in the treatment of a proliferative disease, particularly a cancer or a tumor.

The cancer to be treated is preferably a KRAS G12C mutant cancer.

The cancer or tumor to be treated by administration of the solid forms of the invention include a cancer or tumor which is selected from the group consisting of lung cancer (including lung adenocarcinoma, non-small cell lung cancer and squamous cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma), appendiceal cancer, small-bowel cancer, esophageal cancer, hepatobiliary cancer (including liver cancer and bile duct carcinoma), bladder cancer, ovarian cancer and a solid tumor, particularly when the cancer or tumor harbors a KRAS G12C mutation. Cancers of unknown primary site but showing a KRAS G12C mutation may also benefit from treatment with the solid forms of the of the invention.

Particularly preferred cancers include non-small cell lung cancer, colorectal cancer, pancreatic cancer and a solid tumor.

In another aspect, the invention concerns a method of treating and/or preventing a proliferative disease, particularly a cancer (e.g., non-small cell lung cancer, colorectal cancer, pancreatic cancer and a solid tumor), said method comprising administering a therapeutically effective amount of a crystalline form as defined in the aspects described herein and their corresponding embodiments to a patient in need of such a treatment.

In a further aspect, the invention provides the use of a crystalline compound of the invention for the preparation of a medicament for treating a cancer or tumor, optionally wherein the cancer or tumor is KRAS G12C mutant.

### EXAMPLES

### Example 1: Preparation of 1-{6-[(4M)-4-(5-Chloro-6-methyl-1H-indazol-4-yl)-5-methyl-3-(1-methyl-1H-indazol-5-yl)-1 pyrazol-1-yl]-2-azaspiro[3.3]heptan-2-yl}prop-2-en-1-one (Compound A

A synthesis of 1-{6-[(4M)-4-(5-Chloro-6-methyl-1H-indazol-4-yl)-5-methyl-3-(1-methyl-1-indazol-5-yl)-1H-pyrazol-1-yl]-2-azaspiro[3.3]heptan-2-yl}prop-2-en-1-one (Compound A) is as described below. Compound A is also known by the name “a (R)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one”.

### General Methods and Conditions:

Temperatures are given in degrees Celsius. If not mentioned otherwise, all evaporations are performed under reduced pressure, typically between about 15 mm Hg and 100 mm Hg (=20-133 mbar).

Abbreviations used are those conventional in the art.

Mass spectra were acquired on LC-MS, SFC-MS, or GC-MS systems using electrospray, chemical and electron impact ionization methods with a range of instruments of the following configurations: Waters Acquity UPLC with Waters SQ detector or Mass spectra were acquired on LCMS systems using ESI method with a range of instruments of the following configurations: Waters Acquity LCMS with PDA detector. [M+H]⁺ refers to the protonated molecular ion of the chemical species.

NMR spectra were run with Bruker Ultrashield™400 (400 MHz), Bruker Ultrashield™600 (600 MHz) and BrukerAscend™400 (400 MHz) spectrometers, both with and without tetramethylsilane as an internal standard. Chemical shifts (−values) are reported in ppm downfield from tetramethylsilane, spectra splitting pattern are designated as singlet (s), doublet (d), triplet (t), quartet (q), multiplet, unresolved or more overlapping signals (m), broad signal (br). Solvents are given in parentheses. Only signals of protons that are observed and not overlapping with solvent peaks are reported.

Celite: Celite® (the Celite corporation)=filtering aid based on diatomaceous earth

Phase separator: Biotage-Isolute phase separator-(Part number: 120-1908-F for 70 mL and part number: 120-1909-J for 150 mL)

SiliaMetS® Thiol: SiliCYCLE thiol metal scavenger-(R51030B, Particle Size: 40-63 μm).

X-ray powder diffraction (XRPD) patterns described herein were according to two methods.

### XRPD Method 1

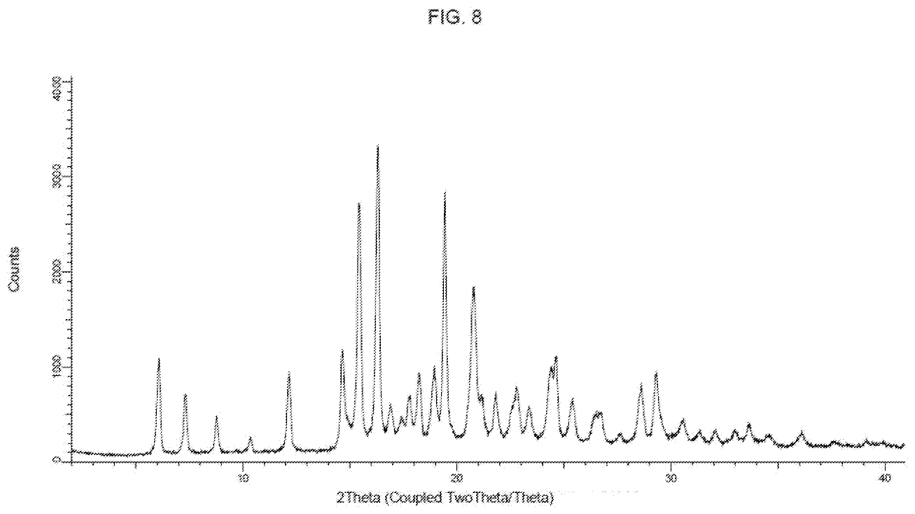

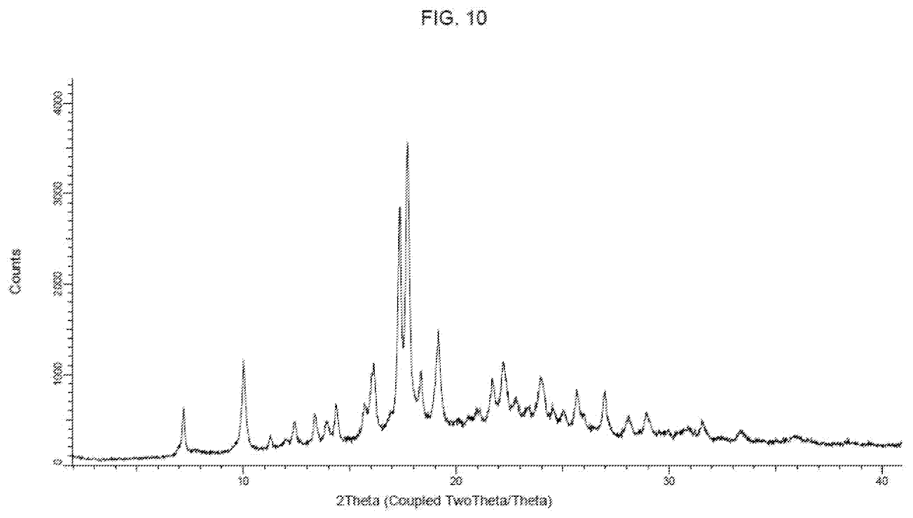

The following method was used to analyze samples obtained in Example 2a to 2e—FIGS. 1 to 5, (Hydrate HA, IPA solvate, ethanol solvate, methanol solvate, propylene glycol solvate of Compound A), Example 3—FIG. 8 (Modification C of Compound A) and Example 5—FIG. 10 (Hydrate HC of Compound A).

X-ray powder diffraction (XRPD) patterns described herein can be obtained using a Bruker Advance D8 in reflection geometry. Powder samples were analyzed using a zero background Si flat sample holder. The radiation was Cu Kα (λ=1.5418 Å). Patterns were measured between 2° and 40° 2theta.

Sample amount: 5-10 mg

Sample holder: zero background Si flat sample holder

XRPD parameters:

| | |
|---|---|
| Instrument | Bruker D8 Advance |
| Detector | LYNXEYE (1D mode), open angle: 2.948°, |
| | scan mode: continuous scan |
| Radiation | CuKα (0.15418 nm) |
| Monochromator | Nickel filter |
| X-ray generator power | 40 kV, 40 mA |
| Goniometer radius | 280 mm |
| Step size | 0.0164°(2-theta value) |
| Time per step | 0.3 second per step |
| Scan range | 2° to 40° (2-theta value) |
| Scan time | About 768 seconds |
| Slits | Primary: fixed illuminated sample size 10 mm; |
| | secondary: open angle 2.2°, axial soller: 2.5° |

### XRPD Method 2

The following method was used to analyze samples obtained in the preparation of n-propanol solvate, 1-butanol solvate, L-lactic acid solvate (Form G), L-lactic acid solvate (Form F), and Example 4 (Hydrate HB of Compound A)

X-ray powder diffraction (XRPD) patterns described herein can be obtained as follows using a Bruker D2 in reflection geometry. Powder samples were analyzed using a zero background Si flat sample holder. The radiation was Cu Kα (λ=1.5418). Patterns were measured between 4° and 40° 2theta.

Sample amount: 5-10 mg

Sample holder: zero background Si flat sample holder

XRPD parameters:

| | |
|---|---|
| Instrument | Bruker D2 |
| Detector | LYNXEYE, scan mode: continuous scan |
| Radiation | CuKα (0.15418 nm) |
| Monochromator | Nickel filter |
| X-ray generator power | 30 kV, 10 mA |
| Goniometer radius | 141 mm |
| Step size | 0.024°(2-theta value) |
| Time per step | 0.15 second per step |
| Scan range | 4° to 40° (2-theta value) |
| Scan time | About 258 seconds |
| Slits | \ |

Degradation products may be measured by HPLC, for example using the paratmeters below.

HPLC

| Instrument | Waters Acquity UPLC | |
|---|---|---|
| Column | ACQUITY BEH C18 | |
| Particle size (μm) | 1.7 | |
| Dimensions (mm) | 2.1 × 100 | |
| Temperature (° C.) | 40 | |
| Flow rate (ml/min) | 0.50 | |
| Injection volume (μl) | 0.5 | |
| Sample solvent | Acetonitrile/Water (50:50) | |
| Sample concentration (μg/ml) | 400 | |
| Detection wavelength (nm) | 210 | |
| Mobile phase A | 0.05% TFA in 95% water/5% acetonitrile | |
| Mobile phase B | 0.05% TFA in 95% acetonitrile/5% water | |
| Run time (min) | 14 | |
| Gradient | Minutes | % B |
| | Initial | 5 |
| | 2.0 | 5 |
| | 9.0 | 60 |
| | 10.0 | 95 |
| | 11.0 | 95 |
| | 11.1 | 5 |
| | 13.0 | 5 |

### Instrumentation

Microwave: All microwave reactions were conducted in a Biotage Initiator, irradiating at 0-400 W from a magnetron at 2.45 GHz with Robot Eight/Robot Sixty processing capacity, unless otherwise stated.

UPLC-MS and MS analytical Methods: Using Waters Acquity UPLC with Waters SQ detector.

UPLC-MS-1: Acquity HSS T3; particle size: 1.8 μm; column size: 2.1×50 mm; eluent A: H₂O+0.05% HCOOH+3.75 mM ammonium acetate; eluent B: CH₃CN+0.04% HCOOH; gradient: 5 to 98% B in 1.40 min then 98% B for 0.40 min; flow rate: 1 mL/min; column temperature: 60° C.

UPLC-MS-3: Acquity BEH C18; particle size: 1.7 μm; column size: 2.1×50 mm; eluent A: H₂O+4.76% isopropanol+0.05% HCOOH+3.75 mM ammonium acetate; eluent B: isopropanol+0.05% HCOOH; gradient: 1 to 98% B in 1.7 min then 98% B for 0.1 min; flow rate: 0.6 mL/min; column temperature: 80° C.

UPLC-MS-4: Acquity BEH C18; particle size: 1.7 μm; column size: 2.1×100 mm; eluent A: H₂O+4.76% isopropanol+0.05% HCOOH+3.75 mM ammonium acetate; eluent B: isopropanol+0.05% HCOOH; gradient: 1 to 60% B in 8.4 min then 60 to 98% B in 1 min; flow rate: 0.4 mL/min; column temperature: 80° C.

UPLC-MS-6: Acquity BEH C18; particle size: 1.7 μm; column size: 2.1×50 mm; eluent A: H₂O+0.05% HCOOH+3.75 mM ammonium acetate; eluent B: isopropanol+0.05% HCOOH; gradient: 5 to 98% B in 1.7 min then 98% B for 0.1 min; flow rate: 0.6 mL/min; column temperature: 80° C.

Preparative Methods:

Chiral SFC Methods:

C-SFC-1: column: Amylose-C NEO 5 μm; 250×30 mm; mobile phase; flow rate: 80 mL/min; column temperature: 40° C.; back pressure: 120 bar.

C-SFC-3: column: Chiralpak AD-H 5 μm; 100×4.6 mm; mobile phase; flow rate: 3 mL/min; column temperature: 40° C.; back pressure: 1800 psi.

### Abbreviations

| Abbreviation | Description |
|---|---|
| AcCN, ACN | acetonitrile |
| Ac₂O | acetic anhydride |
| AcOH | acetic acid |
| AIBN | 2,2′-azobis(2-methylpropionitrile) |
| aq. | aqueous |
| Ar | argon |
| B₂Pin₂ | 4,4,4′,4′,5,5,5′,5′-Octamethyl-2,2′-bi(1,3,2-dioxaborolane) |
| BPR | back pressure |
| brine | saturated aqueous sodium chloride |
| n-BuLi | n-butyl lithium |
| conc. | concentrated |
| DAST | N,N-diethyl-1,1,1-trifluoro-λ⁴-sulfanamine |
| DCE | dichloroethane |
| DCM | dichloromethane |
| DEA | diethylamine |
| DHP | 3,4-dihydropyran |
| DIPEA | N,N-diisopropylethylamine, N-ethyl-N-isopropylpropan- |
| | 2-amine |
| DMA | N,N-dimethylacetamide |
| DMAP | N,N-dimethylpyridin-4-amine |
| DMF | N,N-Dimethylformamide |
| DMSO | dimethylsulfoxide |
| DMSO-d₆ | hexadeuterodimethyl sulfoxide |
| dppf | 1,1′-bis(diphenylphosphanyl) ferrocene |
| ee | enantiomeric excess |
| ESI | electrospray ionization |
| ESI-MS | electrospray ionization mass spectroscopy |
| EtOAc | ethyl acetate |
| GBq | gigabecquerel |
| h | Hour (s) |
| HPLC | high-performance liquid chromatography |
| IPA | 2-propanol |
| KOAc | potassium acetate |
| L/mL/μL | litre/millilitre/microlitre |
| LC-MS | liquid chromatography and mass spectroscopy |
| or LCMS | |
| M | molar |
| MeOH | methanol |
| min (mins) | minute or minutes |
| MTBE | methyl tert-butyl ether |
| MS | mass spectroscopy |
| MW, mw | microwave |
| m/z | mass to charge ratio |
| N | normality |
| N₂ | nitrogen |
| NaOtBu | Sodium tert-butoxide |
| NBS | N-bromosuccinimide |
| NCS | N-chlorosuccinimide |
| NIS | N-iodosuccinimide |
| NEt₃, | triethylamine |
| Et₃N, TEA | |
| PDA | Photodiode array detector |
| NMR | nuclear magnetic resonance |
| Pd(PPh₃)₄ | tetrakis(triphenylphosphane)palladium(0) |
| iPrMgCl | Isopropylmagnesium chloride |
| PTSA | p-toluenesulfonic acid |
| RM | reaction mixture |
| RP | reversed phase |
| Rt | retention time |
| RT | room temperature |
| RuPhos | 2-dicyclohexylphosphino-2′,6′-diisopropoxybiphenyl |
| RuPhos- | (2-dicyclohexylphosphino-2′,6′-diisopropoxy-1,1′- |
| Pd-G3 | biphenyl)[2-(2′-amino-1,1′-biphenyl)]palladium(II) |
| | methanesulfonate |
| Sat. | saturated |
| SFC | supercritical fluid chromatography |
| SQ | Single-quadrupole |
| TBAF | Tetrabutylammonium fluoride |
| tBME, TBME, | tert-butyl methyl ether |
| TBMe | |
| TBq | terabecquerel |
| t-BuOH | tert-butanol |
| tBuXPhos- | tBuXPhos-Pd-G3, [(2-Di-tert-butylphosphino-2′,4′,6′- |
| Pd-G3 | triisopropyl-1,1′-biphenyl)-2-(2′-amino-1,1′- |
| | biphenyl)] palladium(II) methanesulfonate |
| TFA | trifluoroacetic acid |
| THF | tetrahydrofuran |
| TLC | thin-layer chromatography |
| T₃P | propylphosphonic anhydride |
| TsCl | tosyl chloride, 4-Methylbenzene-1-sulfonyl chloride |
| UPLC | ultra-performance liquid chromatography |
| XPhos | 2-dicyclohexylphosphino-2′,4′,6′-triisopropylbiphenyl |
| XPhos-Pd-G3 | (2-dicyclohexylphosphino-2′,4′,6′-triisopropyl-1,1′- |
| | biphenyl)[2-(2′-amino-1,1′-biphenyl)]palladium(II) |
| | methanesulfonate |

All starting materials, building blocks, reagents, acids, bases, dehydrating agents, solvents, and catalysts utilized to prepare the compounds of the present invention are either commercially available or can be produced by organic synthesis methods known to one of ordinary skill in the art. Furthermore, the compounds of the present invention can be produced by organic synthesis methods known to one of ordinary skill in the art as shown in the following examples.

The structures of all final products, intermediates and starting materials are confirmed by standard analytical spectroscopic characteristics, e.g., MS, IR, NMR. The absolute stereochemistry of representative examples of the preferred (most active) atropisomers has been determined by analyses of X-ray crystal structures of complexes in which the respective compounds are bound to the KRASG12C mutant. In all other cases where X-ray structures are not available, the stereochemistry has been assigned by analogy, assuming that, for each pair, the atropisomer exhibiting the highest activity in the covalent competition assay has the same configuration as observed by X-ray crystallography for the representative examples mentioned above. The absolute stereochemistry is assigned according to the Cahn-Ingold-Prelog rule.

### Synthesis of Intermediate C1: tert-butyl 6-(3-bromo-4-(5-chloro-6-methyl-1-(tetrahydro-2H-pyran-2-yl)-1H-indazol-4-yl)-5-methyl-1H-pyrazol-1-yl)-2-azaspiro[3.3]heptane-2-carboxylate

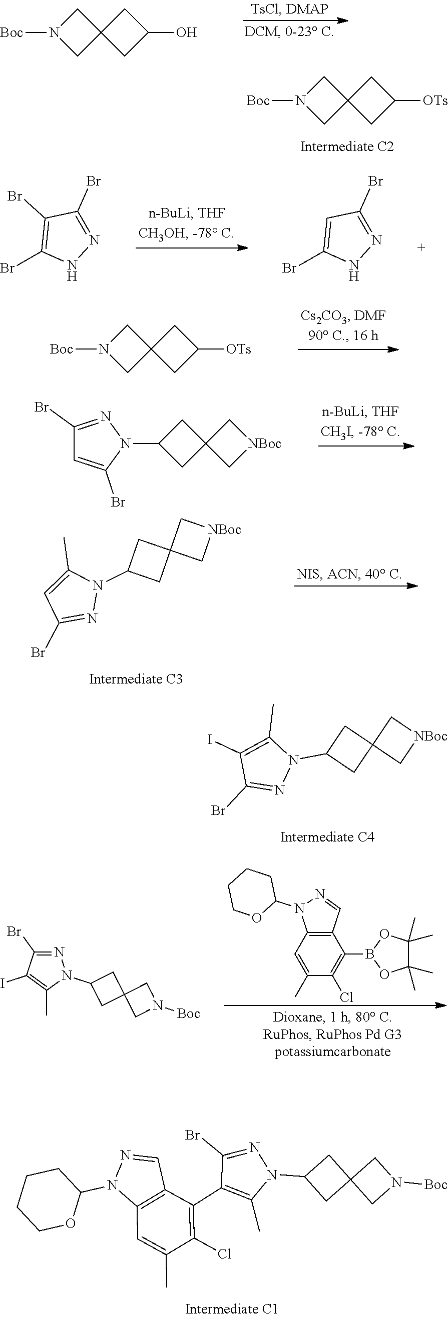

### Step C.1: tert-butyl 6-(tosyloxy)-2-azaspiro[3.3]heptane-2-carboxylate (Intermediate C2

To a solution of tert-butyl 6-hydroxy-2-azaspiro[3.3]heptane-2-carboxylate [CAS No.: 1147557-97-8] (2.92 kg, 12.94 mmol) in DCM (16.5 L) were added DMAP (316.12 g, 2.59 mol) and TsCl (2.96 kg, 15.52 mol) at 20° C.-25° C. To the reaction mixture was added dropwise Et₃N (2.62 kg, 25.88 mol) at 10° C.-20° C. The reaction mixture was stirred 0.5 h at 5° C.-15° C. and then was stirred 1.5 h at 18° C.-28° C. After completion of the reaction, the reaction mixture was concentrated under vacuum. To the residue was added NaCl (5% in water, 23 L) followed by extraction with EtOAc (23 L). The combined aqueous layers were extracted with EtOAc (10 L×2). The combined organic layers were washed with NaHCO₃ (3% in water, 10 L×2)) and concentrated under vacuum to give the title compound. ¹H NMR (400 MHz, DMSO-d₆) δ 7.81-7.70 (m, 2H), 7.53-7.36 (m, 2H), 4.79-4.62 (m, 1H), 3.84-3.68 (m, 4H), 2.46-2.38 (m, 5H), 2.26-2.16 (m, 2H), 1.33 (s, 9H). UPLC-MS-1: Rt=1.18 min; MS m/z [M+H]⁺; 368.2.

### Step C.2: 3,5-dibromo-1H-pyrazole

To a solution of 3,4,5-tribromo-1H-pyrazole [CAS No.: 17635-44-8] (55.0 g, 182.2 mmol) in anhydrous THF (550 mL) was added at −78° C. n-BuLi (145.8 mL, 364.5 mmol) dropwise over 20 min maintaining the internal temperature at −78° C./−60° C. The RM was stirred at this temperature for 45 min. Then the reaction mixture was carefully quenched with MeOH (109 mL) at −78° C. and stirred at this temperature for 30 min. The mixture was allowed to reach to 0° C. and stirred for 1 h. Then, the mixture was diluted with EtOAc (750 mL) and HCl (0.5 N, 300 mL) was added. The layers were concentrated under vacuum. The crude residue was dissolved in DCM (100 mL), cooled to −50° C. and petroleum ether (400 mL) was added. The precipitated solid was filtered and washed with n-hexane (250 mL×2) and dried under vacuum to give the title compound. ¹H NMR (400 MHz, DMSO-d₆) δ 13.5 (br s, 1H), 6.58 (s, 1H).

### Step C.3: tert-butyl 6-(3,5-dibromo-1H-pyrazol-1-yl)-2-azaspiro[3.3]heptane-2-carboxylate

To a solution of tert-butyl 6-(tosyloxy)-2-azaspiro[3.3]heptane-2-carboxylate (Intermediate C2) (Step C.1, 900 g, 2.40 mol) in DMF (10.8 L) was added Cs₂CO₃ (1988 g, 6.10 mol) and 3,5-dibromo-1H-pyrazole (Step C.2, 606 g, 2.68 mol) at 15° C. The reaction mixture was stirred at 90° C. for 16 h. The reaction mixture was poured into ice-water/brine (80 L) and extracted with EtOAc (20 L). The aqueous layer was re-extracted with EtOAc (10 L×2). The combined organic layers were washed with brine (10 L), dried (Na₂SO₄), filtered, and concentrated under vacuum. The residue was triturated with dioxane (1.8 L) and dissolved at 60° C. To the light yellow solution was slowly added water (2.2 L), and recrystallization started after addition of 900 mL of water. The resulting suspension was cooled down to 0° C., filtered, and washed with cold water. The filtered cake was triturated with n-heptane, filtered, then dried under vacuum at 40° C. to give the title compound. ¹H NMR (400 MHz, DMSO-d₃) δ 6.66 (s, 1H), 4.86-4.82 (m, 1H), 3.96-3.85 (m, 4H), 2.69-2.62 (m, 4H), 1.37 (s, 9H); UPLC-MS-3: Rt=1.19 min; MS m/z [M+H]⁺; 420.0/422.0/424.0.

### Step C4: tert-butyl 6-(3-bromo-5-methyl-1H-pyrazol-1-yl)-2-azaspiro[3.3]heptane-2-carboxylate (Intermediate C3

To a solution of tert-butyl 6-(3,5-dibromo-1H-pyrazol-1-yl)-2-azaspiro[3.3]heptane-2-carboxylate (Step C.3, 960 g, 2.3 mol) in THF (9.6 L) was added n-BuLi (1.2 L, 2.5 mol) dropwise at −80° C. under an inert atmosphere. The reaction mixture was stirred 10 min at −80° C. To the reaction mixture was then added dropwise iodomethane (1633 g, 11.5 mol) at −80° C. After stirring for 5 min at −80° C., the reaction mixture was allowed to warm up to 18° C. The reaction mixture was poured into sat. aq. NH₄Cl solution (4 L) and extracted with DCM (10 L). The separated aqueous layer was re-extracted with DCM (5 L) and the combined organic layers were concentrated under vacuum. The crude product was dissolved in 1,4-dioxane (4.8 L) at 60° C., then water (8.00 L) was added dropwise slowly. The resulting suspension was cooled to 17° C. and stirred for 30 min. The solid was filtered, washed with water, and dried under vacuum to give the title compound. ¹H NMR (400 MHz, DMSO-de) 56.14 (s, 1H), 4.74-4.66 (m, 1H), 3.95-3.84 (m, 4H), 2.61-2.58 (m, 4H), 2.20 (s, 3H), 1.37 (s, 9H); UPLC-MS-1: Rt=1.18 min; MS m/z [M+H]⁺; 356.1/358.1.

### Step C.5: tert-butyl 6-(3-bromo-4-iodo-5-methyl-1H-pyrazol-1-yl)-2-azaspiro[3.3]heptane-2-carboxylate (Intermediate C4

To a solution of tert-butyl 6-(3-bromo-5-methyl-1H-pyrazol-1-yl)-2-azaspiro[3.3]heptane-2-carboxylate (Intermediate C3) (Step C.4, 350 g, 0.980 mol) in acetonitrile (3.5 L) was added NIS (332 g, 1.47 mol) at 15° C. The reaction mixture was stirred at 40° C. for 6 h. After completion of the reaction, the reaction mixture was diluted with EtOAc (3 L) and washed with water (5 L×2). The organic layer was washed with Na₂SO₃ (10% in water, 2 L), with brine (2 L), was dried (Na₂SO₄), filtered, and concentrated under vacuum to give the title compound. ¹H NMR (400 MHz, DMSO-d₆) δ 4.81-4.77 (m, 1H), 3.94-3.83 (m, 4H), 2.61-5.59 (m, 4H), 2.26 (s, 3H), 1.37 (s, 9H); UPLC-MS-1: Rt=1.31 min; MS m/z [M+H]⁺; 482.0/484.0.

### Step C.6: tert-butyl 6-(3-bromo-4-(5-chloro-6-methyl-1-(tetrahydro-2H-pyran-2-yl)-1H-indazol-4-yl)-5-methyl-1H-pyrazol-1-yl)-2-azaspiro[3.3]heptane-2-carboxylate (Intermediate C1

To a stirred suspension of tert-butyl 6-(3-bromo-4-iodo-5-methyl-1H-pyrazol-1-yl)-2-azaspiro[3.3]heptane-2-carboxylate (Intermediate C4) (Step C.5, 136 g, 282 mmol) and 5-chloro-6-methyl-1-(tetrahydro-2H-pyran-2-yl)-4-(4,4,5,5-tetramethyl-1,3,2-dioxaborolan-2-yl)-1H-indazole (Intermediate D1, 116 g, 310 mmol) in 1,4-dioxane (680 mL) was added aqueous K₃PO₄ (2M, 467 mL, 934 mmol) followed by RuPhos (13.1 g, 28.2 mmol) and RuPhos-Pd-G3 (14.1 g, 16.9 mmol). The reaction mixture was stirred at 80° C. for 1 h under inert atmosphere. After completion of the reaction, the reaction mixture was poured into 1 M aqueous NaHCO₃ solution (1 L) and extracted with EtOAc (1 L×3). The combined organic layers were washed with brine (1 L×3), dried (Na₂SO₄), filtered, and concentrated under vacuum. The crude residue was purified by normal phase chromatography (eluent: Petroleum ether/EtOAc from 1/0 to 0/1) to give a yellow oil. The oil was dissolved in petroleum ether (1 L) and MTBE (500 mL), then concentrated in vacuo to give the title compound. ¹H NMR (400 MHz, DMSO-d₆) δ 7.81 (s, 1H), 7.66 (s, 1H), 5.94-5.81 (m, 1H), 4.90-4.78 (m, 1H), 3.99 (br s, 2H), 3.93-3.84 (m, 3H), 3.81-3.70 (m, 1H), 2.81-2.64 (m, 4H), 2.52 (s, 3H), 2.46-2.31 (m, 1H), 2.11-1.92 (m, 5H), 1.82-1.67 (m, 1H), 1.64-1.52 (m, 2H), 1.38 (s, 9H); UPLC-MS-3: Rt=1.30 min; MS m/z [M+H]⁺; 604.1/606.1.

### Synthesis of Intermediate D1: 5-chloro-6-methyl-1-(tetrahydro-2H-pyran-2-yl)-4-(4,4,5,5-tetramethyl-1,3,2-dioxaborolan-2-yl)-1H-indazole

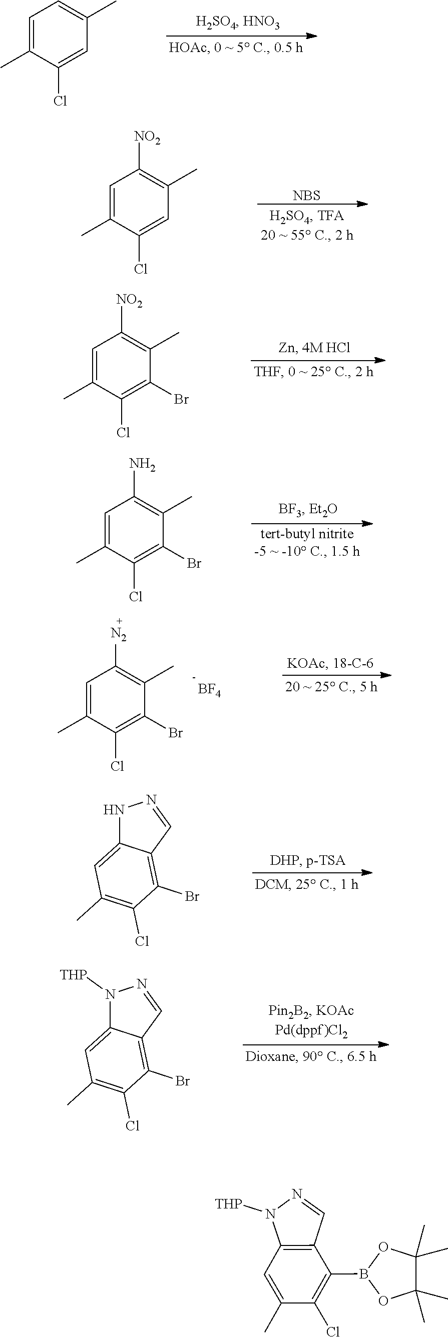

### Step D.1: 1-chloro-2,5-dimethyl-4-nitrobenzene

To an ice-cooled solution of 2-chloro-1,4-dimethylbenzene (3.40 kg, 24.2 mol) in AcOH (20.0 L) was added H₂SO₄ (4.74 kg, 48.4 mol, 2.58 L) followed by a dropwise addition (dropping funnel) of a cold solution of HNO₃ (3.41 kg, 36.3 mol, 2.44 L, 67.0% purity) in H₂SO₄ (19.0 kg, 193. mol, 10.3 L). The reaction mixture was then allowed to stir at 0-5° C. for 0.5 h. The reaction mixture was poured slowly into crushed ice (35.0 L) and the yellow solid precipitated out. The suspension was filtered and the cake was washed with water (5.00 L×5) to give a yellow solid which was suspended in MTBE (2.00 L) for 1 h, filtered, and dried to give the title compound as a yellow solid. ¹H NMR (400 MHz, CDCl₃) δ 7.90 (s, 1H), 7.34 (s, 1H), 2.57 (s, 3H), 2.42 (s, 3H).

### Step D.2: 3-bromo-2-chloro-1,4-dimethyl-5-nitrobenzene

To a cooled solution of 1-chloro-2,5-dimethyl-4-nitrobenzene (Step D.1, 2.00 kg, 10.8 mol) in TFA (10.5 L) was slowly added concentrated H₂SO₄ (4.23 kg, 43.1 mol, 2.30 L) and the reaction mixture was stirred at 20° C. NBS (1.92 kg, 10.8 mol) was added in small portions and the reaction mixture was heated at 55° C. for 2 h. The reaction mixture was cooled to 25° C., then poured into crushed ice solution to obtain a pale white precipitate which was filtered through vacuum, washed with cold water and dried under vacuum to give the title compound as a yellow solid which was used without further purification in the next step. ¹H NMR (400 MHz, CDCl₃) δ 7.65 (s, 1H), 2.60 (s, 3H), 2.49 (s, 3H).

### Step D.3: 3-bromo-4-chloro-2,5-dimethylaniline

To a ice-cooled solution of 3-bromo-2-chloro-1,4-dimethyl-5-nitrobenzene (Step D.2, 2.75 kg, 10.4 mol) in THF (27.5 L) was added HCl (4M, 15.6 L) then Zn (2.72 kg, 41.6 mol) in small portions. The reaction mixture was allowed to stir at 25° C. for 2 h. The reaction mixture was basified by addition of a sat. aq. NaHCO₃ solution (until pH=8). The mixture was diluted with EtOAc (2.50 L) and stirred vigorously for 10 min and then filtered through a pad of celite. The organic layer was separated and the aqueous layer was re-extracted with EtOAc (300 L×4). The combined organic layers were washed with brine (10.0 L), dried (Na₂SO₄), filtered and concentrated under vacuum to give the title compound as a yellow solid which was used without further purification in the next step. ¹H NMR (400 MHz, DMSO-d₆) δ 6.59 (s, 1H), 5.23 (s, 2H), 2.22 (s, 3H), 2.18 (s, 3H).

### Step D.4: 3-bromo-4-chloro-2,5-dimethylbenzenediazonium tetrafluoroborate

BF₃·Et₂O (2.00 kg, 14.1 mol, 1.74 L) was dissolved in DCM (20.0 L) and cooled to −5 to−10° C. under nitrogen atmosphere. A solution of 3-bromo-4-chloro-2,5-dimethylaniline (Step D.3, 2.20 kg, 9.38 mol) in DCM (5.00 L) was added to above reaction mixture and stirred for 0.5 h. Tert-butyl nitrite (1.16 kg, 11.3 mol, 1.34 L) was added dropwise and the reaction mixture was stirred at the same temperature for 1.5 h. TLC (petroleum ether:EtOAc=5:1) showed that starting material (R_{f}=0.45) was consumed completely. MTBE (3.00 L) was added to the reaction mixture to give a yellow precipitate, which was filtered through vacuum and washed with cold MTBE (1.50 L×2) to give the title compound as a yellow solid which was used without further purification in the next step.

### Step D.5: 4-bromo-5-chloro-6-methyl-1H-indazole

To 18-Crown-6 ether (744 g, 2.82 mol) in chloroform (20.0 L) was added KOAc (1.29 kg, 13.2 mol) and the reaction mixture was cooled to 20° C. Then 3-bromo-4-chloro-2,5-dimethylbenzenediazonium tetrafluoroborate (Step D.4, 3.13 kg, 9.39 mol) was added slowly. The reaction mixture was then allowed to stir at 25° C. for 5 h. After completion of the reaction, the reaction mixture was poured into ice cold water (10.0 L), and the aqueous layer was extracted with DCM (5.00 L×3). The combined organic layers were washed with a sat. aq. NaHCO₃ solution (5.00 L), brine (5.00 L), dried (Na₂SO₄), filtered and concentrated under vacuum to give the title compound as a yellow solid. ¹H NMR (600 MHz, CCl₃) δ 10.42 (br s, 1H), 8.04 (s, 1H), 7.35 (s, 1H), 2.58 (s, 3H). UPLC-MS-1: Rt=1.02 min; MS m/z [M+H]⁺; 243/245/247.

### Step D.6: 4-bromo-5-chloro-6-methyl-1-(tetrahydro-2H-pyran-2-yl)-1H-indazole

To a solution of PTSA (89.8 g, 521 mmol) and 4-bromo-5-chloro-6-methyl-1H-indazole (Step D.5, 1.28 kg, 5.21 mol) in DCM (12.0 L) was added DHP (658 g, 7.82 mol, 715 mL) dropwise at 25° C. The mixture was stirred at 25° C. for 1 h. After completion the reaction, the reaction mixture was diluted with water (5.00 L) and the organic layer was separated. The aqueous layer was re-extracted with DCM (2.00 L). The combined organic layers were washed with a sat. aq. NaHCO₃ solution (1.50 L), brine (1.50 L), dried over Na₂SO₄, filtered and concentrated under vacuum. The crude residue was purified by normal phase chromatography (eluent: Petroleum ether/EtOAc from 100/1 to 10/1) to give the title compound as a yellow solid. ¹H NMR (600 MHz, DMSO-de) δ 8.04 (s, 1H), 7.81 (s, 1H), 5.88-5.79 (m, 1H), 3.92-3.83 (m, 1H), 3.80-3.68 (m, 1H), 2.53 (s, 3H), 2.40-232 (in, 1H), 2.06-1.99 (m, 1H), 1.99-1.93 (m, 1H), 1.77-1.69 (m, 1H), 1.60-1.56 (m, 2H). UPLC-MS-6: Rt=1.32 min; MS m/z [M+H]⁺; 329.0/331.0/333.0

### Step D.7: 5-chloro-6-methyl-1-(tetrahydro-2H-pyran-2-yl)-4-(4,4,5,5-tetramethyl-1,3,2-dioxaborolan-2-yl)-1H-indazole (Intermediate D.1

A suspension of 4-bromo-5-chloro-6-methyl-1-(tetrahydro-2H-pyran-2-yl)-1H-indazole (Step 0.6, 450 g, 1.37 mol), KOAc (401 g, 4.10 mol) and B₂Pin₂ (520 g, 2.05 mol) in 1,4-dioxane (3.60 L) was degassed with nitrogen for 0.5 h. Pd(dppf)Cl₂·CH₂Cl₂ (55.7 g, 68.3 mmol) was added and the reaction mixture was stirred at 90° C. for 6 h. The reaction mixture was filtered through diatomite and the filter cake was washed with EtOAc (1.50 L×3). The mixture was concentrated under vacuum to give a black oil which was purified by normal phase chromatography (eluent: Petroleum ether/EtOAc from 100/1 to 10/1) to give the desired product as brown oil. The residue was suspended in petroleum ether (250 mL) for 1 h to obtain a white precipitate. The suspension was filtered, dried under vacuum to give the title compound as a white solid. ¹H NMR (400 MHz, CDCl₃) δ 8.17 (d, 1H), 7.52 (s, 1H), 5.69-5.66 (m, 1H), 3.99-3.96 (m, 1H), 3.75-3.70 (m, 1H), 2.51 (d, 4H), 2.21-2.10 (m, 1H), 2.09-1.99 (m, 1H), 1.84-1.61 (m, 3H), 1.44 (s, 12H); UPLC-MS-6: Rt=1.29 min; MS m/z [M+H]⁺; 377.1/379.

### Synthesis of Compound A

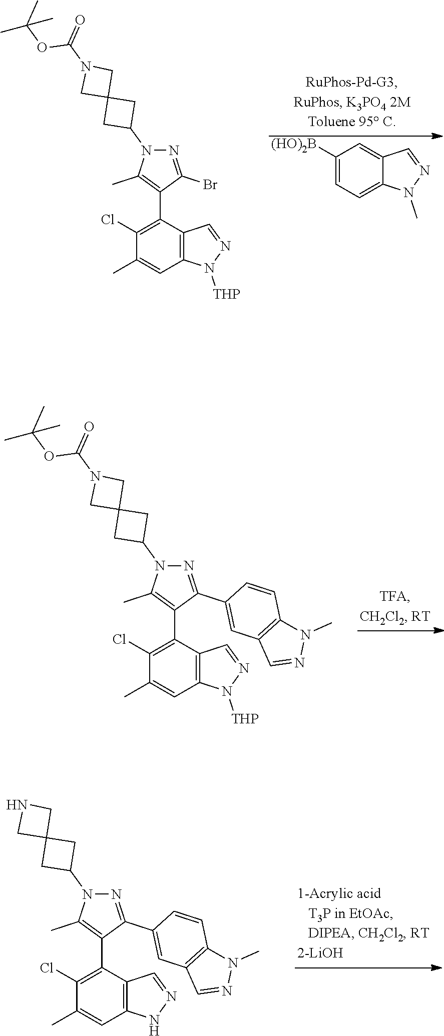

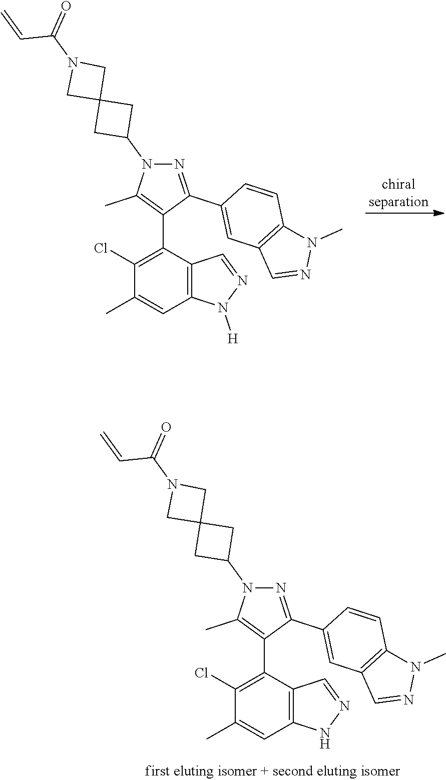

### Step 1: Tert-butyl 6-(4-(5-chloro-6-methyl-1-(tetrahydro-2H-pyran-2-yl)-1H-indazol-4-yl)-5-methyl-3-(1-methyl-1H-indazol-5-yl)-1H-pyrazol-1-yl)-2-azaspiro[3.3]heptane-2-carboxylate

In a 500 mL flask, tert-butyl 6-(3-bromo-4-(5-chloro-6-methyl-1-(tetrahydro-2H-pyran-2-yl)-1H-indazol-4-yl)-5-methyl-1H-pyrazol-1-yl)-2-azaspiro[3.3]heptane-2-carboxylate (Intermediate C1, 10 g, 16.5 mmol), (1-methyl-1H-indazol-5-yl)boronic acid (6.12 g, 33.1 mmol), RuPhos (1.16 g, 2.48 mmol) and RuPhos-Pd-G3 (1.66 g, 1.98 mmol) were suspended in toluene (165 mL) under argon. K₃PO₄ (2M, 24.8 mL, 49.6 mmol) was added and the reaction mixture was placed in a preheated oil bath (95° C.) and stirred for 45 min. The reaction mixture was poured into a sat. aq. NH₄Cl solution and was extracted with EtOAc (×3). The combined organic layers were washed with a sat. aq. NaHCO₃ solution, dried (phase separator) and concentrated under reduced pressure. The crude residue was diluted with THF (50 mL), SiliaMetS® Thiol (15.9 mmol) was added and the mixture swirled for 1 h at 40° C. The mixture was filtered, the filtrate was concentrated and the crude residue was purified by normal phase chromatography (eluent: MeOH in CH₂Cl₂ from 0 to 2%), the purified fractions were again purified by normal phase chromatography (eluent: MeOH in CH₂Cl₂ from 0 to 2%) to give the title compound as a beige foam. UPLC-MS-3: Rt=1.23 min; MS m/z [M+H]⁺; 656.3/658.3.

### Step 2: 5-Chloro-6-methyl-4-(5-methyl-3-(1-methyl-1H-indazol-5-yl)-1-(2-azaspiro[3.3]heptan-6-yl)-1H-pyrazol-4-yl)-1H-indazole

TFA (19.4 mL, 251 mmol) was added to a solution of tert-butyl 6-(4-(5-chloro-6-methyl-1-(tetrahydro-2H-pyran-2-yl)-1H-indazol-4-yl)-5-methyl-3-(1-methyl-1H-indazol-5-yl)-1H-pyrazol-1-yl)-2-azaspiro[3.3]heptane-2-carboxylate (Step 1, 7.17 g, 10.0 mmol) in CH₂Cl₂ (33 mL). The reaction mixture was stirred at RT under nitrogen for 1.5 h. The RM was concentrated under reduced pressure to give the title compound as a trifluoroacetate salt, which was used without purification in the next step. UPLC-MS-3: Rt=0.74 min; MS m/z [M+H]⁺; 472.3/474.3.

### Step 3: 1-(6-(4-(5-Chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one

A mixture of acrylic acid (0.69 mL, 10.1 mmol), propylphosphonic anhydride (50% in EtOAc, 5.94 mL, 7.53 mmol) and DIPEA (21.6 mL, 126 mmol) in CH₂Cl₂ (80 mL) was stirred for 20 min at RT and then added (dropping funnel) to an ice-cooled solution of 5-chloro-6-methyl-4-(5-methyl-3-(1-methyl-1H-indazol-5-yl)-1-(2-azaspiro[3.3]heptan-6-yl)-1H-pyrazol-4-yl)-1H-indazole trifluoroacetate (Step 2, 6.30 mmol) in CH₂Cl₂ (40 mL). The reaction mixture was stirred at RT under nitrogen for 15 min. The RM was poured into a sat. aq. NaHCO₃ solution and extracted with CH═Cl₂ (×3). The combined organic layers were dried (phase separator) and concentrated. The crude residue was diluted with THF (60 mL) and LiOH (2N, 15.7 mL, 31.5 mmol) was added. The mixture was stirred at RT for 30 min until disappearance (UPLC) of the side product resulting from the reaction of the acryloyl chloride with the free NH group of the indazole then was poured into a sat. aq. NaHCO₃ solution and extracted with CH₂Cl₂ (3×). The combined organic layers were dried (phase separator) and concentrated. The crude residue was purified by normal phase chromatography (eluent: MeOH in CH₂Cl₂ from 0 to 5%) to give the title compound. The isomers were separated by chiral SFC (C-SFC-1; mobile phase: CO₂/[IPA+0.1% Et₃N]: 69/31) to give Example 1: 1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one (Compound A) (also known as a (R)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one) as the second eluting peak (white powder, in amorphous form): ¹H NMR (600 MHz, DMSO-d₆) δ 13.1 (s, 1H), 7.89 (s, 1H), 7.59 (s, 1H), 7.55 (s, 1H), 7.42 (m, 2H), 7.30 (d, 1H), 6.33 (in, 1H), 6.12 (in, 1H), 5.68 (n, 1H), 4.91 (m, 1H), 4.40 (s, 1H), 4.33 (s, 1H), 4.11 (s, 1H), 4.04 (s, 1H), 3.95 (s, 3H), 2.96-2.86 (m, 2H), 2.83-2.78 (m, 2H), 2.49 (s, 3H), 2.04 (s, 3H); UPLC-MS-4: Rt=4.22 min; MS m/z [M+H]⁺ 526.3/528.3; C-SFC-3 (mobile phase: CO₂/[IPA+0.1% Et₃N]: 67/33): Rt=2.23 min. The compound of Example 1 is also referred to as “Compound A”.

The atropisomer of Compound A, a(S)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one was obtained as the first eluting peak: C-SFC-3 (mobile phase: CO₂/[IPA+01% Et₃N]: 67/33): Rt=1.55 min.

### Example 2: Synthesis of Hydrate HA of Compound A

### Example 2a: Crystalline Isopropyl Alcohol (IPA) Solvate of Compound A and Crystalline Hydrate (Hydrate HA) Form of Compound A

25 mg of amorphous Compound A (obtained from Example 1 above) was added to 0.1 mL of 2-propanol. The resulting clear solution was stirred at 25° C. for 3 days, after which crystalline solid precipitated out. The solid was collected by centrifugal filtration (i.e. filtration using a centrifuge). The wet cake was characterized as crystalline isopropyl (IPA) solvate of Compound A. Drying of the wet cake at ambient condition overnight provided crystalline Hydrate HA.

Hydrate HA of Compound A was analysed by XRPD (see FIG. 1) and its characteristic peaks are shown in the Table below.

In particular, the most characteristic peaks of the XRPD pattern of the Hydrate HA of Compound A may be selected from one, two, three or four peaks having an angle of refraction 20 values (CuKα λ=1.5418 Å) selected from the group consisting of 8.2°, 11.6°, 12.9° and 18.8°.

| Index | Two-Theta | d value | | Relative intensity | Intensity |
|---|---|---|---|---|---|
| 1 | 8.2° | 10.72 | Å | 100% | Strong |
| 2 | 11.6° | 7.60 | Å | 11% | Weak |
| 3 | 12.1° | 7.30 | Å | 10% | Weak |
| 4 | 12.9° | 6.87 | Å | 14% | Medium |
| 5 | 14.6° | 6.05 | Å | 21% | Medium |
| 6 | 16.2° | 5.47 | Å | 15% | Medium |
| 7 | 18.8° | 4.73 | Å | 28% | Medium |
| 8 | 20.4° | 4.34 | Å | 18% | Medium |
| 9 | 24.1° | 3.69 | Å | 29% | Medium |

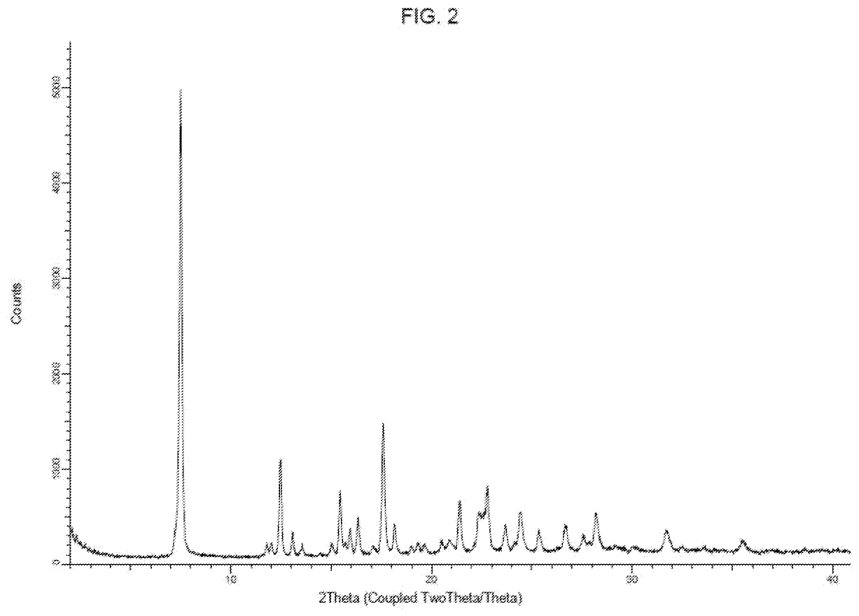

Crystalline IPA solvate form of Compound A was analysed by XRPD (see FIG. 2) and its characteristic peaks are shown in the Table below. In particular, the most characteristic peaks of the XRPD pattern of the crystalline IPA solvate form may be selected from two, or three peaks having an angle of refraction 2θ values (CuKα λ=1.5418 Å) selected from the group consisting of 7.5°, 12.5° and 17.6°.

| Index | Two-Theta | d value | Relative intensity | Intensity |
|---|---|---|---|---|
| 1 | 7.5° | 11.77 | 100% | Strong |
| 2 | 12.5° | 7.08 Å | 20% | Medium |
| 3 | 15.5° | 5.73 Å | 14% | Low |
| 4 | 16.4° | 5.42 Å | 8% | Low |
| 5 | 17.6° | 5.04 Å | 28% | Medium |
| 6 | 21.4° | 4.15 Å | 11% | Low |
| 7 | 24.4° | 3.64 Å | 8% | Low |

### Example 2b: Crystalline Ethanol (EtOH) Solvate of Compound A and Crystalline Hydrate (Hydrate HA) Form of Compound A

25 mg of amorphous Compound A (obtained from Example 1 above) was added to 0.1 mL of ethanol. The resulting clear solution was stirred at 25° C. for 3 days. Crystals of Hydrate HA of Compound A obtained in example 1 was added as seeds to the resulting solution. The resulting suspension was equilibrated for another 1 day, after which a solid precipitated out. The solid was collected by centrifugal filtration. The wet cake was characterized as crystalline ethanol solvate, which after drying at ambient condition overnight, produced Hydrate HA of Compound A.

Alternatively, 3.1 g of Compound A was added to 20 mL of ethanol and the resulting clear solution was stirred at 25° C. for 20 mins. Approximately 50 mg crystalline Hydrate HA (obtained above) were added as seeds, and the resulting mixture was equilibrated at 25° C. for 6 hours. The resulting suspension was filtered, and the wet cake was characterized as crystalline ethanol solvate. The solid was then dried at ambient condition (25° C., 60-70% Relative Humidity (RH)) for 3 days and 2.8 g of Hydrate HA of Compound A were obtained with a yield of 90%.

Crystalline ethanol solvate crystals can also be obtained without the addition of seeds of Hydrate HA. Compound A was suspended in ethanol for at least an hour, after which a solid precipitated out. The solid was collected by centrifugal filtration. The wet cake was characterized as crystalline ethanol solvate, which after drying at ambient condition overnight, produced Hydrate HA crystals.

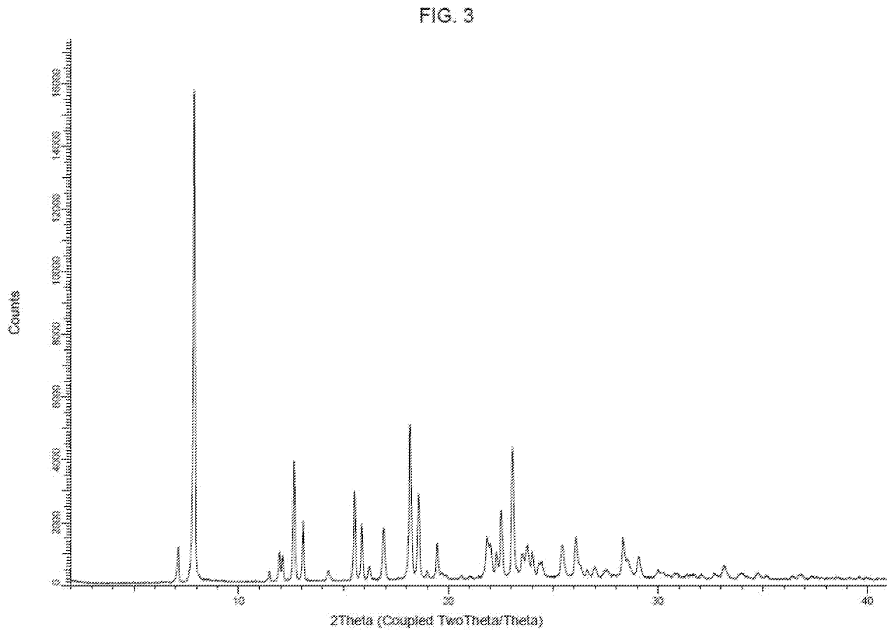

Crystalline ethanol solvate form of Compound A was analysed by XRPD (see FIG. 3) and its characteristic peaks are shown in the Table below.

In particular, the most characteristic peaks of the XRPD pattern of the crystalline ethanol solvate form may be selected from two, or three or four peaks having an angle of refraction 2@ values (CuKα λ=1.5418 Å) selected from the group consisting of 7.9°, 12.7°, 18.2° and 23.1°.

| Index | Two-Theta | d value | | Relative intensity | Intensity |
|---|---|---|---|---|---|
| 1 | 7.9° | 11.20 | Å | 100% | Strong |
| 2 | 12.7° | 6.99 | Å | 24% | Medium |
| 3 | 13.1° | 6.76 | Å | 12% | weak |
| 4 | 15.5° | 5.70 | Å | 18% | weak |
| 5 | 15.9° | 5.58 | Å | 11% | weak |
| 6 | 16.9° | 5.24 | Å | 11% | weak |
| 7 | 18.2° | 4.88 | Å | 32% | medium |
| 8 | 18.6° | 4.77 | Å | 17% | weak |
| 9 | 23.1° | 3.85 | Å | 26% | Medium |

### Example 2c-1: Alternative Preparation of Crystalline Hydrate (Hydrate HA) Preparation from the Crystalline Ethanol Solvate of Compound A

Hydrate HA of Compound A may be prepared by first forming the ethanol solvate of Compound A by adding Compound A to a solvent mixture of dichloromethane and ethanol, removing the dichloromethane (e.g., by distillation), recovering the ethanol solvate crystalline material from the resulting suspension, e.g., by filtration, and then drying the wet cake of ethanol solvate crystals at a high temperature, e.g. in the range of 50 to 60° C., under a water vapor atmosphere to form Hydrate HA crystals.

The following procedure can be used.

4.00 kg of Compound A and 0.040 kg 1% citric acid were dissolved in a solvent mixture of 11 kg dichloromethane and 9 kg ethanol. The resulting mixture was filtered. The filtrate was collected and 39 kg of ethanol was added to the filtrate. The resulting solution was distilled under vacuum, at a temperature of less than 55° C. (i.e. the jacket temperature (JT) was kept at ≤55° C.) to remove the dichloromethane. 28 kg of distillate were collected in the receiving tank. A further 26 kg of ethanol was added to the residual solution in the reactor. The resulting solution was again concentrated under vacuum with the jacket temperature set at ≤55° C. and 25 kg of distillate were collected in the receiving tank.

26 kg of ethanol was added to the residual solution in the reactor. The residual solution in the reactor was then heated up to a temperature between 60 to 70° C. After 15 minutes at that temperature, the mixture was cooled to a lower temperature of 50 to 60° C. 0.010 kg of ethanol solvate of Compound A were added as seed crystals. The resulting suspension was stirred for at least 30 minutes, cooled to a temperature of 30-40° C. and then stirred for at least 60 minutes. The suspension was concentrated under vacuum at a temperature of less than 55° C. (JT≤55° C.) to remove ethanol and to recover 20 kg of distillate in the receiving tank.

The resulting mixture in the distillation reactor was heated to a temperature of 60-70° C. After 15 minutes at that temperature, the resulting mixture was cooled to 0-10° C., stirred for at least 6 hours and then filtered. The wet wake was washed with ethanol.

The wet cake (which was characterized as the ethanol solvate) was then dried in an oven under controlled vacuum at 50° C. with a water vapor atmosphere. The pressure in the oven varied between 30 and 60 mbar. Drying was carried out until the ethanol residue left in the crystals was at an acceptable level, e.g. less than 2000 ppm. Crystals of Hydrate HA were obtained.

### Example 2c-2: Alternative Preparation of Crystalline Hydrate (Hydrate HA) Preparation from a Crystalline Alcohol Solvate of Compound A

Isopropanol solvate crystals (100 g) were dissolved in a mixture of tetrahydrofuran (THF, 366.5 g) and ethanol (122.7 g) at a temperature in the range of 35-40° C. The resulting mixture was filtered and the filter was rinsed with the solvent mixture of THF/ethanol. The filtrate was cooled to ambient temperature (e.g. in the range 20 to 30° C.). Ethanol (66.7 g) was added to the filtrate. Seed crystals of Hydrate HA crystals were then added as a suspension (0.50 g in 2.50 g ethanol). The resulting mixture was agitated in a solicitor for 30 minutes to produce crystals of the ethanol solvate of Compound A.

The THF solvent was then removed by vacuum distillation. The volume in the distillation flask or reactor was kept constant by the addition of ethanol.

The resulting mixture in the distillation reactor was then agitated at ambient temperature for a short period (e.g. 30 minutes), heated to a temperature ranging between 30 to 40° C. for one hour and then cooled to 0-10° C. The mixture was then agitated for a further 2 hours, filtered, and washed with cold ethanol. After that, the wet cake (which was characterized as the ethanol solvate) was dried in an oven under controlled vacuum at 50° C. with a water vapor atmosphere. The pressure in the oven varied between 40 and 60 mbar. Hydrate HA crystals were obtained in 89% yield.

### Example 2d: Alternative Preparation of Crystalline Hydrate (Hydrate HA) Preparation

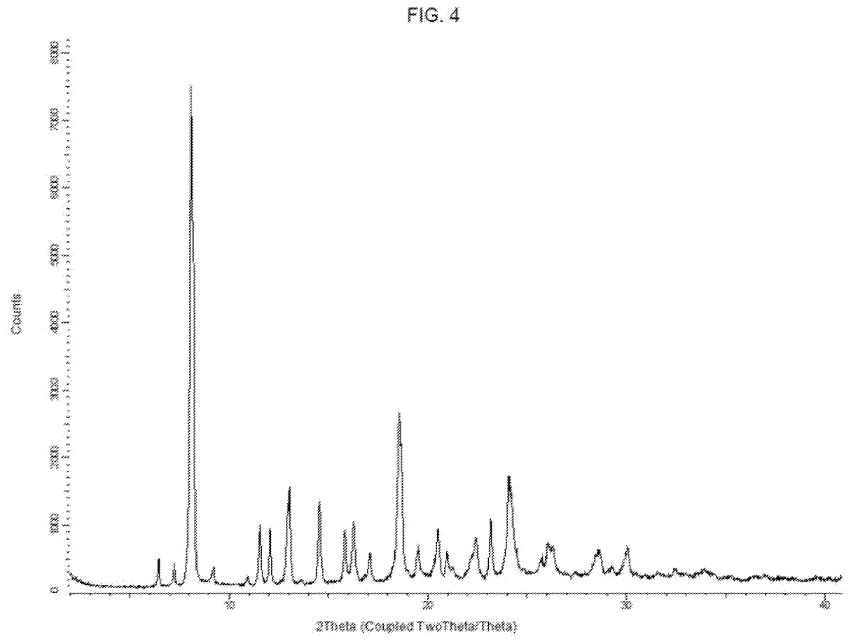

25 mg of Compound A (obtained from Example 1 above) was added to 0.1 mL of methanol. The resulting clear solution was stirred at 25° C. for 3 days. Hydrate HA crystals obtained in example 1 were added as seeds to the resulting solution. The resulting suspension was equilibrated for another 1 day, after which a solid precipitated out. The solid was collected by centrifugal filtration and dried at ambient condition overnight. The wet cake was analyzed by XRPD and found to contain a mixture of the methanol solvate and Hydrate HA of Compound A (see FIG. 4). After drying at ambient condition overnight, the wet cake produced crystalline hydrate (Hydrate HA) of Compound A.

### Example 2e: Crystalline Propylene Glycol Solvate of Compound A and Crystalline Hydrate (Hydrate HA

25 mg of Compound A (obtained from Example 1 above) was added to 0.1 mL of propylene glycol. The resulting suspension was stirred at 50° C. for 1 week. The solid was collected by centrifugal filtration. The wet cake obtained after filtration was characterized as crystalline propylene glycol solvate. After drying of the cake at ambient condition for 1 week, crystalline Hydrate HA was obtained.

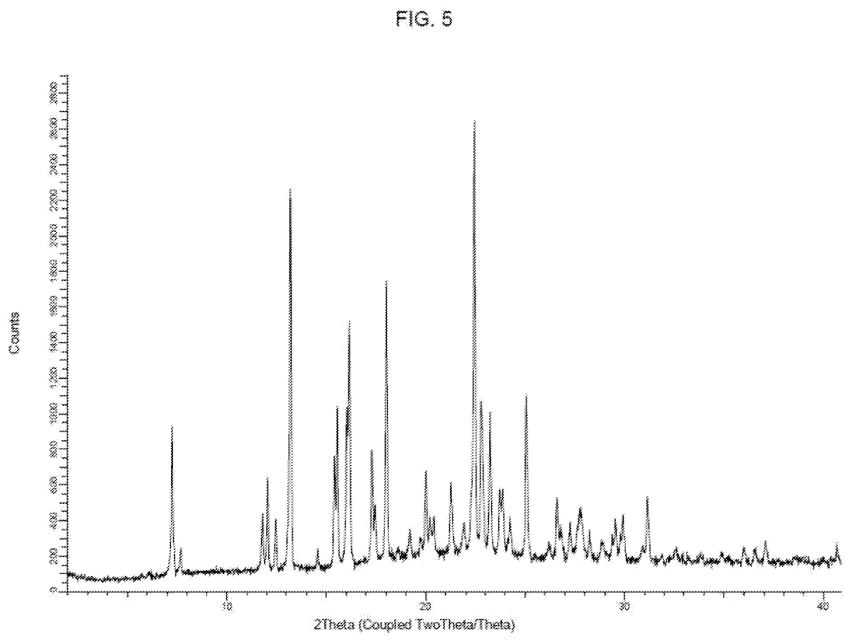

Crystalline propylene glycol solvate form of Compound A was analysed by XRPD (see FIG. 5) and its characteristic peaks are shown in the Table below. In particular, the most characteristic peaks of the XRPD pattern of the crystalline propylene glycol solvate form may be selected from two, or three or four peaks having an angle of refraction 2θ values (CuKα λ=1.5418 Å) selected from the group consisting of 7.3°, 13.2°, 18.0° and 22.5°.

| Index | Two-Theta | d value | | Relative intensity | Intensity |
|---|---|---|---|---|---|
| 1 | 7.3° | 12.15 | Å | 34% | weak |
| 2 | 13.2° | 6.70 | Å | 87% | Strong |
| 3 | 15.6° | 5.69 | Å | 37% | weak |
| 4 | 16.2° | 5.48 | Å | 56% | Medium |
| 5 | 18.0° | 4.92 | Å | 64% | Medium |
| 6 | 22.5° | 3.96 | Å | 100% | Strong |
| 7 | 22.8° | 3.90 | Å | 35% | weak |
| 8 | 23.2° | 3.83 | Å | 33% | weak |
| 9 | 25.1° | 3.55 | Å | 37% | weak |

### Example 2f: Crystalline 1-Butanol Solvate of Compound A and Hydrate (Hydrate HA) of Compound A

150 mg of Compound A (obtained from Example 1 above) were added to 1 to 2 mL of 1-butanol. The resulting suspension was stirred at room temperature for 1 day. The solid was collected by centrifugal filtration. The wet cake obtained after filtration was characterized as crystalline 1-butanol solvate. The wet cake solid was allowed to stand at 50° C. and at 75 RH % for 1 day to give the crystalline Hydrate HA.

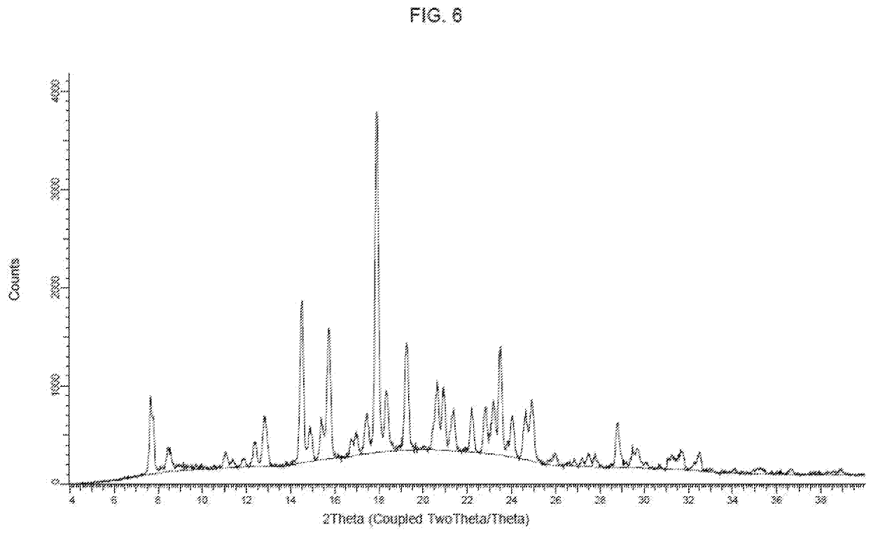

Crystalline 1-butanol solvate form of Compound A was analysed by XRPD (see FIG. 6) and its characteristic peaks are shown in the Table below. In particular, the most characteristic peaks of the XRPD pattern of the crystalline 1-butanol solvate form may be selected from two, or three or four peaks having an angle of refraction 26 values (CuKα λ=1.5418 Å) selected from the group consisting of 7.7°, 14.5° 17.9° and 19.3°.

| Index | Two-Theta | d value | | Relative intensity | Intensity |
|---|---|---|---|---|---|
| 1 | 7.7° | 11.44 | Å | 18% | weak |
| 2 | 12.8° | 6.89 | Å | 14% | weak |
| 3 | 14.5° | 6.10 | Å | 47% | Medium |
| 4 | 15.7° | 5.63 | Å | 38% | Medium |
| 5 | 17.9° | 4.95 | Å | 100% | Medium |
| 6 | 19.3° | 4.61 | Å | 31% | Medium |
| 7 | 21.3° | 4.16 | Å | 11% | weak |
| 8 | 22.2° | 4.00 | Å | 11% | weak |
| 9 | 24.0° | 3.70 | Å | 12% | weak |
| 10 | 28.8° | 3.10 | Å | 13% | weak |

### Example 2: Crystalline n-Propanol Solvate and Hydrate HA of Compound A

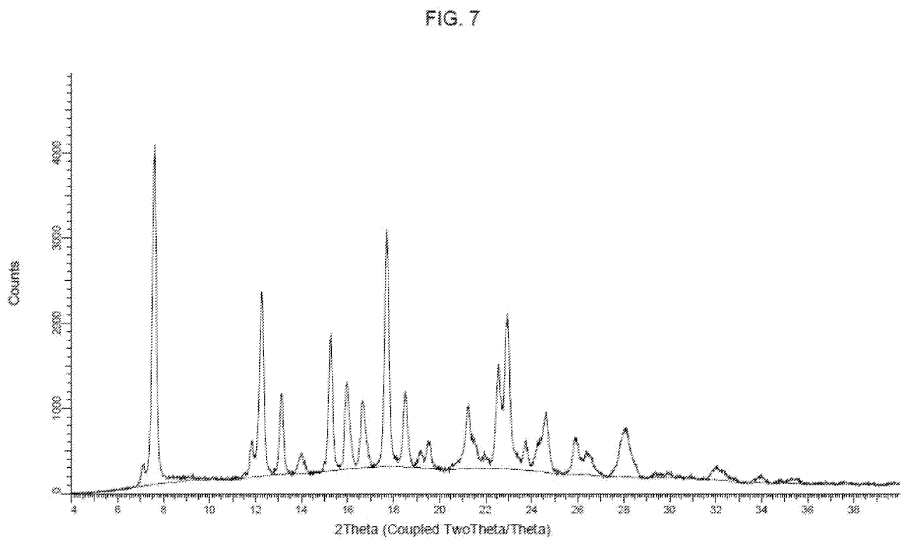

90 mg of Compound A (obtained from Example 1 above) was added to 1˜2 mL of n-propanol. The resulting suspension was stirred at room temperature for 1 day. The solid was collected by centrifugal filtration. The wet cake obtained after filtration was analyzed by XRPD (FIG. 7) and characterized as crystalline n-propanol solvate. The wet cake solid was allowed to stand under 50° C./75 RH % for 1 day, and the crystalline hydrate (Hydrate HA) was obtained.

Crystalline n-propanol solvate form of Compound A was analysed by XRPD (see FIG. 7) and the most characteristic peaks are shown in the Table below. In particular, the most characteristic peaks of the XRPD pattern of the crystalline n-Propanol solvate form may be selected from two, or three or four peaks having an angle of refraction 2θ values (CuKα λ=1.5418 Å) selected from the group consisting of 7.6°, 15.3°, 17.7° and 13.5°.

| Index | Two-Theta | d value | | Relative intensity | Intensity |
|---|---|---|---|---|---|
| 1 | 7.6° | 11.61 | Å | 100% | Strong |
| 2 | 12.3° | 7.21 | Å | 56% | Medium |
| 3 | 13.1° | 6.73 | Å | 24% | weak |
| 4 | 15.3° | 5.80 | Å | 39% | Medium |
| 5 | 16.0° | 5.54 | Å | 25% | weak |
| 6 | 16.7° | 5.31 | Å | 20% | weak |
| 7 | 17.7° | 5.00 | Å | 72% | Strong |
| 8 | 18.5° | 4.79 | Å | 23% | weak |
| 9 | 28.1° | 3.18 | Å | 15% | weak |

### Example 3: Synthesis of Modification C of Compound A

50 mg Hydrate HA crystals (obtained as described in Example 2 above) and 11.6 mg benzoic acid (as additive) were added to 2 ml of MTBE (methyl tert-butyl ether). The resulting suspension was stirred at room temperature for several days. The solid was collected by centrifugal filtration. The wet cake was characterized by XPRD as crystalline anhydrate (Modification C of low crystallinity) form of Compound A.

Alternatively, Modification C can also be obtained as follows.

450 mg Hydrate HA crystals (obtained as described in Example 2 above) were added to 8 ml of ethyl acetate/heptane (volume/volume, 1:1) mixture. 60 mg acetic acid was added to 1 ml ethyl acetate. The solution containing Compound A and the acetic acid solution were mixed together. The resulting material was stirred at room temperature for 34 days. The solid was collected by centrifugal filtration. The wet cake was characterized as crystalline anhydrate (Modification C of low crystallinity) form of Compound A.

45.8 mg of isopropyl alcohol solvate crystals of Compound A were added to 0.2 ml of 3-pentanone. The resulting material was heated to 50° C. and stirred to obtain a clear solution. 0.6 ml of MTBE was added to the solution. The resulting mixture (obtained either as a suspension or a gel) was stirred at room temperature to 40° C. for 7 days. The solid was collected by centrifugal filtration. The wet cake was characterized as crystalline anhydrate (Modification C of medium crystallinity) form of Compound A.

Modification C crystalline material of high crystallinity may be obtained as follows.

30 ml ethyl acetate (EtOAc) were added to 2.06 g of amorphous Compound A and stirred to obtain a clear solution at 55° C. The clear solution was filtered to remove undissolved material. Another 2 ml EtOAc was used to wash the vessel and transferred to the bulk solution. The solution was stirred at 53° C., and 24 mg Modification C solid crystals were added as seeds. After stirring at 53° C. for about 19 h, the clear solution became cloudier. Then 12 ml heptane was added in 6 h, and the temperature of the mixture was held at 53° C. for 4 h. The resulting suspension was cooled to 20° C. in 3 h and held at 20° C. for 11 h. The resulting suspension was filtered and washed with 10 ml EtOAc/heptane (v/v, 7/3). The solid was dried at 50° C. for 5 h under vacuum. 1.75 g solid of Modification C was obtained.

Modification C of Compound A was analysed by XRPD (see FIG. 8) and its characteristic peaks are shown in the Table below. In particular, the most characteristic peaks of the XRPD pattern of the crystalline hydrate (Modification C) form may be selected from one, two, three or four peaks having an angle of refraction 2θ values (CuKα λ=1.5418 Å) selected from the group consisting of 6.1°, 12.2°, 16.3°, and 19.4°.

| Index | Two-Theta | d value | | Relative intensity | Intensity |
|---|---|---|---|---|---|
| 1 | 6.1° | 14.56 | Å | 31% | Weak |
| 2 | 7.3° | 12.08 | Å | 19% | Weak |
| 3 | 8.8° | 10.07 | Å | 12% | Weak |
| 4 | 12.2° | 7.28 | Å | 26% | Weak |
| 5 | 14.7° | 6.04 | Å | 33% | Weak |
| 6 | 15.4° | 5.74 | Å | 82% | Strong |
| 7 | 16.3° | 5.44 | Å | 100% | Strong |
| 8 | 18.2° | 4.86 | Å | 22% | Weak |
| 9 | 19.4° | 4.56 | Å | 79% | Strong |
| 10 | 20.8° | 4.27 | Å | 50% | Medium |
| 11 | 21.8° | 4.07 | Å | 13% | Weak |
| 12 | 25.4° | 3.51 | Å | 13% | Weak |
| 13 | 29.4° | 3.04 | Å | 22% | Weak |

### Example 4: Crystalline Hydrate (Hydrate HB) Form of Compound A

100 mg of Compound A (amorphous) were added to 1 mL of a solvent mixture of methanol and water (6:4 v/v). The resulting suspension was stirred at room temperature to obtain a clear solution. Hydrate HA crystals were added as seed crystals. The resulting mixture was stirred at room temperature for 36 hours. The solids were collected by centrifugal filtration. The wet cake obtained after filtration was analyzed by XPRD and characterized as crystalline hydrate (Hydrate HB) of compound A. The wet cake solid was allowed to stand at 30° C. and 90% RH for 23 hours to afford dry crystalline hydrate (Hydrate HB).

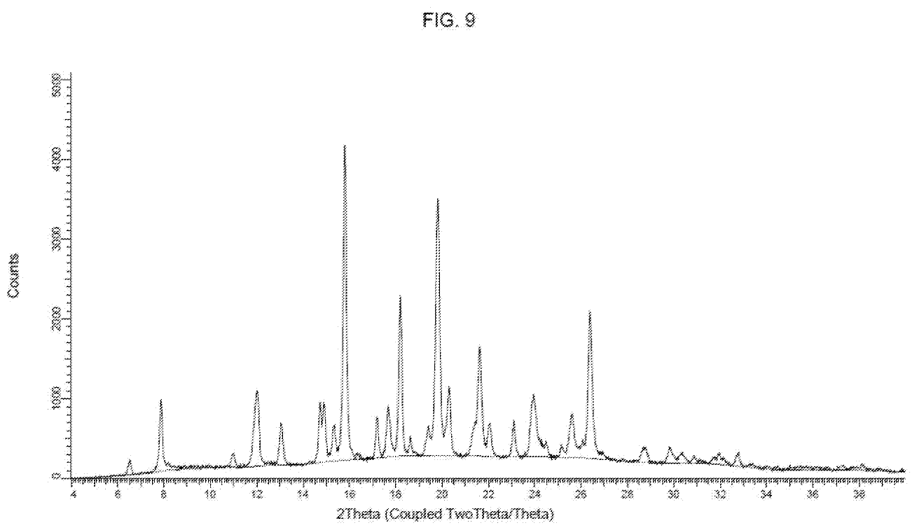

Hydrate HB of Compound A was analysed by XRPD (see FIG. 9) and its characteristic peaks are shown in the Table below. In particular, the most characteristic peaks of the XRPD pattern of the crystalline hydrate (Hydrate HB) form may be selected from two, or three or four peaks having an angle of refraction 2θ values (Cuα λ=1.5418 Å) selected from the group consisting of 7.9°, 15.8°, 18.2°, and 26.4°.

| Index | Two-Theta | d value | | Relative intensity | Intensity |
|---|---|---|---|---|---|
| 1 | 6.5° | 13.55 | Å | 4% | Weak |
| 2 | 7.9° | 11.20 | Å | 23% | Weak |
| 3 | 12.0° | 7.38 | Å | 23% | Weak |
| 4 | 13.1° | 6.77 | Å | 13% | Weak |
| 5 | 15.8° | 5.60 | Å | 100% | Strong |
| 6 | 17.2° | 5.15 | Å | 13% | Weak |
| 7 | 17.7° | 5.01 | Å | 15% | Weak |
| 8 | 18.2° | 4.87 | Å | 50% | Medium |
| 9 | 19.8° | 4.48 | Å | 82% | Strong |
| 10 | 21.6° | 4.11 | Å | 33% | Weak |
| 11 | 23.1° | 3.85 | Å | 11% | Weak |
| 12 | 26.4° | 3.37 | Å | 46% | Medium |

### Example 5: Crystalline Hydrate HC Form of Compound A

Crystals of Hydrate HB of Compound A obtained in Example 4 above were dried in an oven at 50° C. for 17 hours to give crystalline Hydrate HC of Compound A. Hydrate HC of Compound A was analyzed by XRPD (see FIG. 10) and its characteristic peaks are shown in the Table below. In particular, the most characteristic peaks of the XRPD pattern of the crystalline hydrate (Hydrate HC) form may be selected from two, or three or four peaks having an angle of refraction 2θ values (CuKα λ=1.5418 Å) selected from the group consisting of 7.2°, 10.0°, 19.2°, and 27.0°.

| Index | Two-Theta | d value | | Relative intensity | Intensity |
|---|---|---|---|---|---|
| 1 | 7.2° | 12.26 | Å | 16% | Weak |
| 2 | 10.0° | 8.81 | Å | 31% | Weak |
| 3 | 13.4° | 6.60 | Å | 10% | Weak |
| 4 | 14.4° | 6.15 | Å | 13% | Weak |
| 5 | 17.4° | 5.10 | Å | 78% | Strong |
| 6 | 17.7° | 5.00 | Å | 100% | Strong |
| 7 | 19.2° | 4.63 | Å | 32% | Weak |
| 8 | 22.2° | 3.99 | Å | 21% | Weak |
| 9 | 24.0° | 3.70 | Å | 16% | Weak |
| 10 | 27.0° | 3.30 | Å | 15% | Weak |

### Example 6: Crystalline L-Lactic Acid Solvate (Form G) Form of Compound A

69.6 mg isopropyl alcohol solvate crystals of Compound A was added to 0.2 ml of L-lactic acid. The mixture was stirred at room temperature to obtain a clear solution. 1 ml of MTBE was added to the solution. The resulting solution was allowed to stand with no sealed cap under ambient condition. Some solid was observed and collected. The solid was characterized as crystalline form G of the L-lactic acid solvate of Compound A.

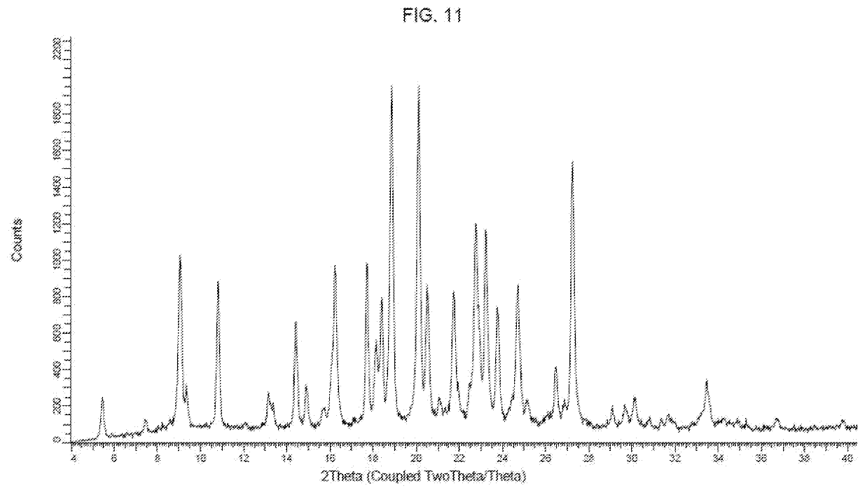

Form G of the L-lactic acid solvate of Compound A was analyzed by XRPD (see FIG. 11) and its characteristic peaks are shown in the Table below. In particular, the most characteristic peaks of the XRPD pattern of the crystalline hydrate (Hydrate HC) form may be selected from two, or three or four peaks having an angle of refraction 2θ values (CuKα 2=1.5418 Å) selected from the group consisting of 10.8°, 16.2°, 8.9°, and 27.3°.

| Index | Two-Theta | d value | | Relative intensity | Intensity |
|---|---|---|---|---|---|
| 1 | 5.5° | 16.18 | Å | 12% | Weak |
| 2 | 9.1° | 9.75 | Å | 52% | Medium |
| 3 | 10.8° | 8.17 | Å | 43% | Medium |
| 4 | 14.4° | 6.13 | Å | 31% | Weak |
| 5 | 16.2° | 5.46 | Å | 47% | Medium |
| 6 | 17.7° | 5.00 | Å | 47% | Medium |
| 7 | 18.9° | 4.70 | Å | 100% | Strong |
| 8 | 20.1° | 4.41 | Å | 97% | Strong |
| 9 | 21.8° | 4.08 | Å | 36% | Weak |
| 10 | 23.8° | 3.74 | Å | 32% | Weak |
| 11 | 24.7° | 3.60 | Å | 38% | Weak |
| 12 | 27.3° | 3.27 | Å | 74% | Strong |

### Example 7: Crystalline L-Lactic Acid Solvate (Form F) Form of Compound A

226 mg of isopropyl alcohol solvate of Compound A crystals were added to 0.35 ml of L-lactic acid and 3 ml of MTBE mixture. Crystalline form G of the L-lactic acid solvate of Compound A were added as seed crystals. The resulting mixture was stirred at room temperature for 3 days. The solid was collected by centrifugal filtration. The wet cake obtained after filtration was characterized as crystalline form F of compound A. The wet cake solid was dried under vacuum at 45° C. for 2.5 h to obtain dry crystalline form F of the L-lactic acid solvate of Compound A.

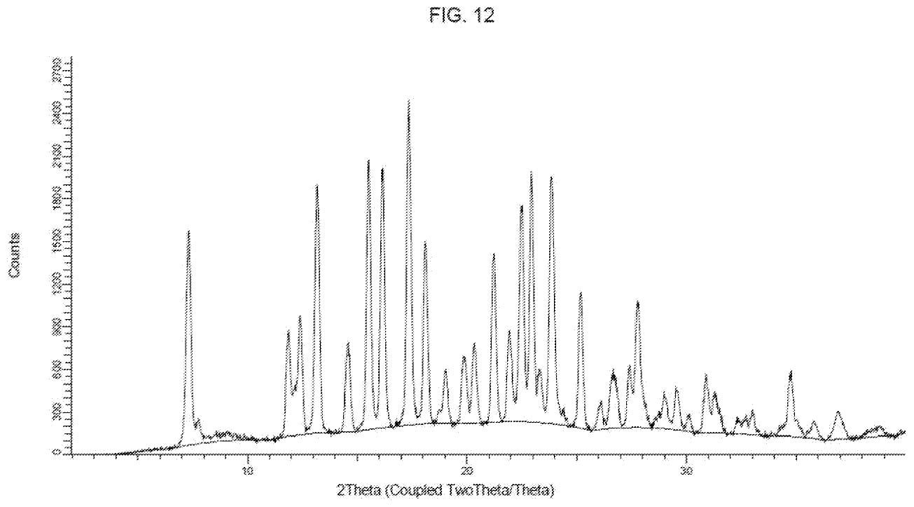

Form F of the L-lactic acid solvate of Compound A was analyzed by XRPD (see FIG. 12) and its characteristic peaks are shown in the Table below. In particular, the most characteristic peaks of the XRPD pattern of the crystalline L-lactic acid solvate form may be selected from two, or three or four peaks having an angle of refraction 2θ values (CuKα λ=1.5418 Å) selected from the group consisting of 13.2°, 17.4°, 21.2°, and 25.2°.

| Index | Two-Theta | d value | | Relative intensity | Intensity |
|---|---|---|---|---|---|
| 1 | 7.3° | 12.07 | Å | 68% | Strong |
| 2 | 13.2° | 6.71 | Å | 79% | Strong |
| 3 | 14.6° | 6.07 | Å | 28% | Weak |
| 4 | 15.5° | 5.70 | Å | 85% | Strong |
| 5 | 16.2° | 5.48 | Å | 79% | Strong |
| 6 | 17.4° | 5.11 | Å | 100% | Strong |
| 7 | 18.1° | 4.89 | Å | 57% | Medium |
| 8 | 19.0° | 4.66 | Å | 16% | Weak |
| 9 | 20.3° | 4.36 | Å | 25% | Weak |
| 10 | 21.2° | 4.18 | Å | 53% | Medium |
| 11 | 23.9° | 3.72 | Å | 77% | Strong |
| 12 | 25.2° | 3.53 | Å | 43% | Medium |

### Example 8: Water Sorption and Desorption Experiments

Water sorption and desorption isotherms were recorded at 25° C., 40° C. or 60° C. using a Dynamic Vapor Sorption (DVS) instrument as follows. The cycle used was as follows: 40-0-95-0-40 (% RH).

| | |
|---|---|
| Instrument | Advantage/Intrinsic |
| Sample mass | Approximately 10 mg |
| Temperature | 25° C. or 40° C. or 60° C. |
| Dm/dt (change in mass v/s time) | 0.002%/min |
| Maximum/minimum stage time | 360 min/60 min |

### Water Sorption and Desorption Isotherm of Modification C

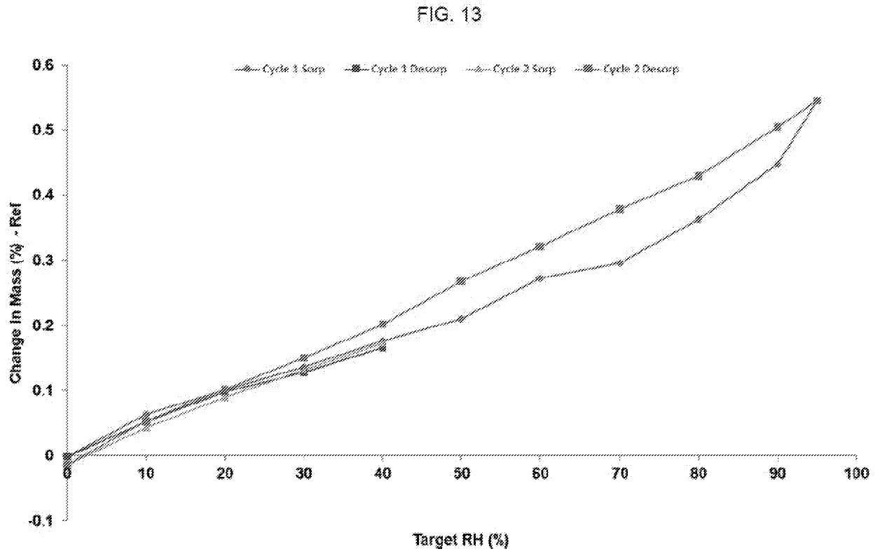

The maximum water uptake of Modification C is about 0.5% at 25° C. and up to 95% RH. Modification C is non hygroscopic. FIG. 13 shows the water sorption-desorption isotherm of Modification C at 25° C., 40-0-95-0-40 (% RH) with dm/dt 0.002%/min.

| | Target | Change In Mass (%) - ref | | |
|---|---|---|---|---|
| | RH (%) | Sorption | Desorption | Hysteresis |
| Cycle 1 | 0.0 | −0.0018 | −0.0018 | |
| | 10.0 | 0.0639 | 0.0522 | −0.0117 |
| | 20.0 | 0.1010 | 0.0984 | −0.0026 |
| | 30.0 | 0.1365 | 0.1282 | −0.0083 |
| | 40.0 | 0.1764 | 0.1658 | −0.0107 |
| | 50.0 | 0.2099 | | |
| | 60.0 | 0.2719 | | |
| | 70.0 | 0.2954 | | |
| | 80.0 | 0.3628 | | |
| | 90.0 | 0.4475 | | |
| | 95.0 | 0.5450 | | |
| Cycle 2 | 0.0 | −0.0143 | −0.0143 | |
| | 10.0 | 0.0434 | 0.0532 | 0.0098 |
| | 20.0 | 0.0893 | 0.1019 | 0.0126 |
| | 30.0 | 0.1314 | 0.1500 | 0.0186 |
| | 40.0 | 0.1725 | 0.2014 | 0.0289 |
| | 50.0 | | 0.2678 | |
| | 60.0 | | 0.3212 | |
| | 70.0 | | 0.3789 | |
| | 80.0 | | 0.4299 | |
| | 90.0 | | 0.5046 | |
| | 95.0 | | 0.5450 | |

### Water Sorption and Desorption Isotherm of Hydrate HA

The water sorption and desorption isotherm of Hydrate HA shows a reversible uptake and release of up to 8% of water at 95% RH. The isotherm is reversible with only small hysteresis between sorption and desorption, which suggests that Hydrate HA is a channel hydrate. Hydrate HA can host up to 2.5 molecules of (corresponding to a water content of 7.9%) depending on the relative humidity.

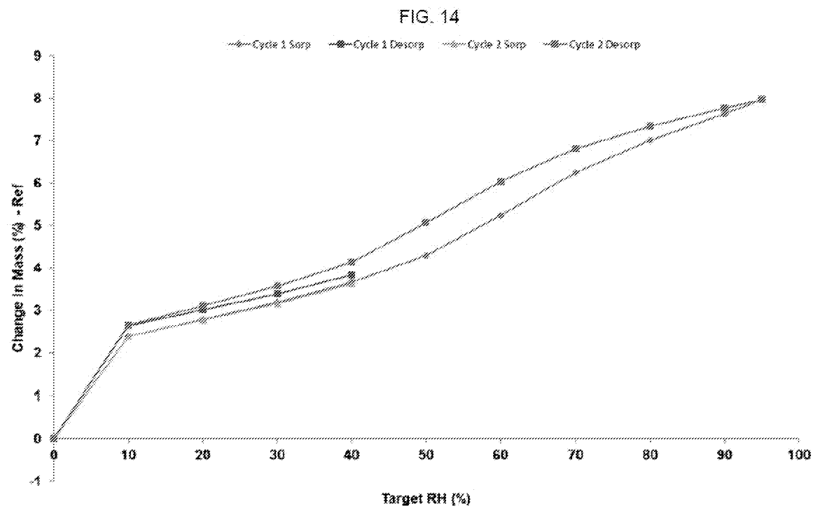

The maximum water uptake of Hydrate HA is about 8% at 25° C. and up to 95% RH. Hydrate HA is hygroscopic FIG. 14 shows the isotherm plot of Hydrate HA of Compound A at 25° C., 40-0-95-0-40 (% RH) with dm/dt 0.002%/min.

| | Target | Change In Mass (%) - ref | | |
|---|---|---|---|---|
| | RH (%) | Sorption | Desorption | Hysteresis |
| Cycle 1 | 0.0 | 0.002 | 0.002 | |
| | 10.0 | 2.390 | 2.638 | 0.248 |
| | 20.0 | 2.782 | 3.008 | 0.226 |
| | 30.0 | 3.184 | 3.381 | 0.198 |
| | 40.0 | 3.660 | 3.838 | 0.177 |
| | 50.0 | 4.295 | | |
| | 60.0 | 5.234 | | |
| | 70.0 | 6.244 | | |
| | 80.0 | 7.007 | | |
| | 90.0 | 7.633 | | |
| | 95.0 | 7.973 | | |
| Cycle 2 | 0.0 | 0.000 | 0.000 | |
| | 10.0 | 2.388 | 2.659 | 0.271 |
| | 20.0 | 2.773 | 3.102 | 0.329 |
| | 30.0 | 3.163 | 3.573 | 0.410 |
| | 40.0 | 3.624 | 4.126 | 0.502 |
| | 50.0 | | 5.062 | |
| | 60.0 | | 6.040 | |
| | 70.0 | | 6.816 | |
| | 80.0 | | 7.346 | |
| | 90.0 | | 7.766 | |
| | 95.0 | | 7.973 | |

### Water Sorption and Desorption Isotherm of Hydrate HB

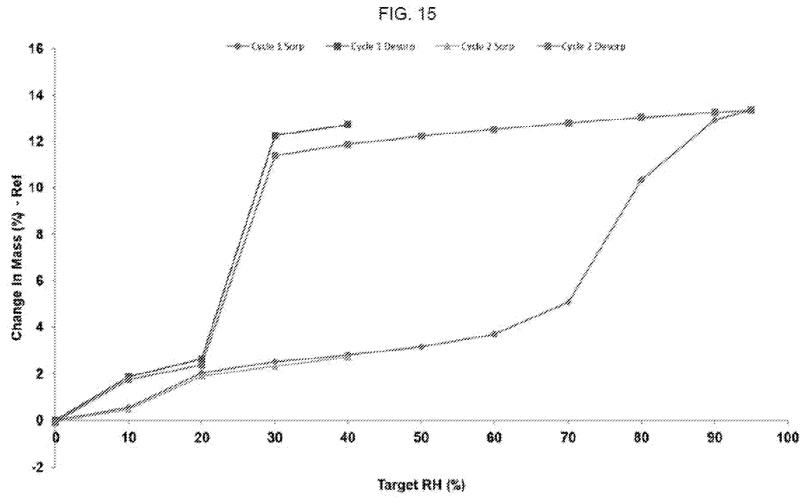

The maximum water uptake of Hydrate HB is about 13% at 25° C. and up to 95% RH. FIG. 15, shows the isotherm plot of Hydrate HB of Compound A at 25° C., 40-0-95-0-40 (% RH) with dm/dt 0.002%/min).

| | Target | Change In Mass (%) - ref | | |
|---|---|---|---|---|
| | RH (%) | Sorption | Desorption | Hysteresis |
| Cycle 1 | 0.0 | 0.00 | 0.00 | |
| | 10.0 | 0.56 | 1.87 | 1.31 |
| | 20.0 | 2.03 | 2.62 | 0.60 |
| | 30.0 | 2.51 | 12.26 | 9.75 |
| | 40.0 | 2.81 | 12.73 | 9.92 |
| | 50.0 | 3.17 | | |
| | 60.0 | 3.69 | | |
| | 70.0 | 5.09 | | |
| | 80.0 | 10.34 | | |
| | 90.0 | 12.94 | | |
| | 95.0 | 13.35 | | |
| Cycle 2 | 0.0 | −0.09 | −0.09 | |
| | 10.0 | 0.47 | 1.74 | 1.27 |
| | 20.0 | 1.88 | 2.37 | 0.49 |
| | 30.0 | 2.32 | 11.38 | 9.07 |
| | 40.0 | 2.69 | 11.87 | 9.18 |
| | 50.0 | | 12.24 | |
| | 60.0 | | 12.53 | |
| | 70.0 | | 12.78 | |
| | 80.0 | | 13.02 | |
| | 90.0 | | 13.24 | |
| | 95.0 | | 13.35 | |

### Example 9: Granulation Simulation Experiments

Granulating solvent was added drop wise to the solid form being tested until the solid form was wetted sufficiently. The wet substance was ground manually. The solid form was evaluated for degree of crystallinity or form change by e.g., XRPD analysis and/or DSC analysis. Granulating solvents were water, pH 4.7, 50 mM acetate buffer, and pH 6.8, 50 mM phosphate buffer.

TABLE
Granulation simulation experiments

| Solvent for | XRPD of | XRPD of | XRPD of |
|---|---|---|---|
| granulation | Hydrate HA | Hydrate H_{B} | Modification C |
| Water | No form | Change to Hydrate | No form |
| | change | H_{C}, crystallinity | change |
| | | decreased | |
| pH 4.7, 50 mM | No form | Change to Hydrate | No form |
| acetate buffer | change | H_{C}, crystallinity | change |
| | | decreased | |
| pH 6.8, 50 mM | No form | Change to Hydrate | No form |
| phosphate buffer | change | H_{C}, crystallinity | change |
| | | decreased | |

It can be seen that Hydrate HA and Modification C are suitable for further processing into pharmaceutical dosage forms.

### Example 10: Differential Scanning Calorimetry

Differential scanning calorimetry (DSC) was carried out using the following instrument parameters

| Instrument | TA Discovery DSC | |
|---|---|---|
| Temperature range | 0° C. −300° C. | |
| Heating rate | 10 | K/min |
| Nitrogen flow | 50 | mL/min |

The DSC of Hydrate HA of Compound A shows two endothermic events with peak temperatures at around 28° C. and 78° C., when heated at 10 K/min, which are most likely associated to dehydration and melting. Upon further heating the sample shows a glass transition at about 138° C.

The DSC of the tetrahydrate H_{L} shows endothermic events with an onset temperature at around 44° C., when heated at 10 K/min, which are most likely associated to dehydration. Upon further heating the sample shows another small endothermic event at about 141° C., which may be associated to the melting of Modification B or to a relaxation phenomenon at the glass transition.

Modification C is a stable anhydrous form. A sample with 76% crystallinity exhibited a melting point at about 215° C. when heated in a DSC at 10K/min in a sample pan with a pin hole. Melting was associated by decomposition.

### Example 11: Equilibration Studies

Equilibration studies were performed as follows. About 25 mg of a given solid form were equilibrated with 0.1-0.5 mL of solvent for at least 2 weeks at 25° C. Suspensions were filtered and dried for 10 min in the air. The solid part was investigated by XRPD (X-ray powder diffraction). Additional investigations may also be optionally performed (e.g. DSC, TG, IR, SEM).

Equilibration studies of Hydrate HA of Compound A at 25° C. did not show the formation of a new crystalline form. Equilibration studies at 50° C. and 70° C. showed the formation of several solvates in cyclohexane, ethanol, isopropanol or 1,2-propanediol.

### Example 12: Bulk Stability Studies

The stability of the crystalline form was investigated as follows. Bulk samples were analysed, e.g. by HPLC and/or XRPD after being exposed to various temperatures and residual humidities.

| | XRPD of | XRPD of | XRPD of |
|---|---|---|---|
| Test conditions | Hydrate HA | Hydrate H_{B} | Modification C |
| 1 week 50° C., | No form change, | No form change, | No change. |
| 75% RH | crystallinity | crystallinity | .No |
| | decreased No | decreased. No | discoloration |
| | discoloration | discoloration | |
| % Degradation | 1.45% | 3.97% | 0.59% |
| Products, as | | | |
| measured by | | | |
| HPLC | | | |

Degradation Products are analyzed by HPLC They are calculated as area- % products.

1. A crystalline form of the compound, 1-{6-[(4M)-4-(5-chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one, of formula

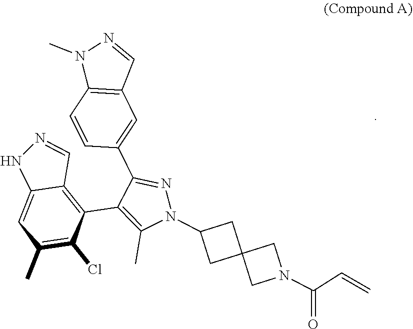

2. A crystalline form according to claim 1 which is selected from Hydrate HA, an alcohol solvate (e.g. an isopropyl alcohol solvate, an ethanol solvate, a methanol solvate, a propylene glycol solvate, a 1-butanol solvate, or an n-propanol solvate), Modification C, Hydrate HB, Hydrate C, and a lactic acid solvate form (e.g., Form G of the L-lactic acid solvate or Form F of L-lactic acid solvate).

3. A crystalline form according to claim 1, 2 or 3, which has an X-ray powder diffraction pattern substantially the same as the X-ray powder diffraction pattern shown in FIG. 1, FIG. 2, FIG. 3, FIG. 4, FIG. 5, FIG. 6, FIG. 7, FIG. 8, FIG. 9, FIG. 10, FIG. 11, or FIG. 12, when measured using CuKα radiation.

4. A crystalline form according to claim 1 or 2 or 3, which is in substantially pure form.

5. A crystalline form according to claim 1, 2, 3 or 4, which is Hydrate HA.

6. A crystalline form according to any one of claims 1 to 5, which has an X-ray powder diffraction pattern with at least one, two, three or four peaks having an angle of refraction 2θ values (CuKα λ=1.5418 Å) selected from the group consisting of 8.2°, 11.6°, 12.9° and 18.8°, measured at a temperature of about 25° C. and an x-ray wavelength, λ, of 1.5418 Å.

7. A crystalline form according to claim 6, wherein the X-ray powder diffraction pattern further contains at least one, two, three, four or five peaks having an angle of refraction 2θ values (CuKα λ=1.5418 Å) selected from the group consisting of 12.1°, 14.6°, 16.2°, 20.4° and 24.1°, measured at a temperature of about 25° C. and an x-ray wavelength, λ, of 1.5418 Å.

8. The crystalline Hydrate HA of the compound according to claim 1, 2 or 3, which has an X-ray powder diffraction pattern substantially the same as the X-ray powder diffraction pattern shown in FIG. 1 when measured using CuKα radiation.

9. A process for the preparation of crystalline form Hydrate HA of Compound A comprising the steps:
(i) suspending Compound A in an alcohol to form the corresponding alcoholic solvate of Compound A in crystalline form;
(ii) separating at least a part of the crystals obtained from the mother liquor;
(iii) optionally washing the isolated crystals; and
(iv) drying the separated crystals (optionally drying under reduced pressure) in a humid atmosphere to form Hydrate HA crystalline form.

10. A process according to claim 9, wherein the alcoholic solvent is selected from methanol, ethanol, 2-propanol, propylene glycol, n-propanol and 1-butanol, and combinations thereof.

11. A process according to claim 9 or 10, wherein drying is carried out at a relative humidity of below 90%.

12. The use of an alcoholic solvate of Compound A in a process for the preparation of Hydrate HA of Compound A.

13. A crystalline form according to claim 1, 2, 3 or 4, which is Modification C.

14. A crystalline form according to any one of claims 1 to 4, or claim 13, which has an X-ray powder diffraction pattern with at least one, two, three or four peaks having an angle of refraction 2θ values (CuKα λ=1.5418 Å) selected from the group consisting of 6.1°, 12.2°, 16.3°, and 19.4°, measured at a temperature of about 25° C. and an x-ray wavelength, λ, of 1.5418 Å.

15. A crystalline form according to claim 14, wherein the X-ray powder diffraction pattern further contains at least one, two, three, four, five, six, seven or eight, or all peaks having an angle of refraction 2θ values (CuKα λ=1.5418 Å) selected from the group consisting of 7.3°, 8.8°, 14.7°, 15.4°, 18.2°, 20.8°, 21.8°, 25.4° and 29.4°, measured at a temperature of about 25° C. and an x-ray wavelength, λ, of 1.5418 Å.

16. The crystalline Modification C of the compound according to claim 13, 14 or 15, which has an X-ray powder diffraction pattern substantially the same as the X-ray powder diffraction pattern shown in FIG. 8 when measured using CuKα radiation.

17. A crystalline form according to claim 1 which is an alcoholic solvate, optionally wherein the solvate is an isopropyl alcohol solvate, an ethanol solvate, a methanol solvate, a propylene glycol solvate, a 1-butanol solvate, or an n-propanol solvate.

18. A crystalline form of claim 1 which is a lactic solvate form (e.g., an L-lactic acid solvate) of Compound A, optionally wherein the lactic acid solvate is Form G of the L-lactic acid solvate or Form F of L-lactic acid solvate.

19. A pharmaceutical composition comprising a crystalline form according to any one of claims 1 to 8, or any one of claims 13 to 18, and at least one pharmaceutically acceptable carrier or diluent.

20. A crystalline form according to any one of claims 1 to 8, or any one of claims 13 to 18, for use as a medicament.

21. A crystalline form according to any one of claims 1 to 8, or any one of claims 13 to 18, for use in the treatment of cancer, especially for KRAS G12C mutant cancer.

22. A crystalline form according to any one of claims 1 to 8, or any one of claims 13 to 18, wherein the cancer is a cancer or tumor which is selected from the group consisting of lung cancer (including lung adenocarcinoma, non-small cell lung cancer and squamous cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma), appendiceal cancer, small-bowel cancer, esophageal cancer, hepatobiliary cancer (including liver cancer and bile duct carcinoma), bladder cancer, ovarian cancer and a solid tumor, particularly when the cancer or tumor harbors a KRAS G12C mutation.

23. Use of a compound according to any one of claims 1 to 8, or any one of claims 13 to 18, for the manufacture of a medicament for the treatment of cancer.

24. Use according to claim 23, wherein the cancer is cancer is a cancer or tumor which is selected from the group consisting of lung cancer (including lung adenocarcinoma, non-small cell lung cancer and squamous cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma), appendiceal cancer, small-bowel cancer, esophageal cancer, hepatobiliary cancer (including liver cancer and bile duct carcinoma), bladder cancer, ovarian cancer and a solid tumor, particularly when the cancer or tumor harbors a KRAS G12C mutation.

25. A method of treatment of cancer, comprising administering to a subject or patient in need thereof a therapeutically effective amount of a crystalline form according to any one of claims 1 to 8, or any one of claims 13 to 18, or a pharmaceutical composition according to claim 19.

26. The method of claim 25, wherein the cancer is a cancer is a cancer or tumor which is selected from the group consisting of lung cancer (including lung adenocarcinoma, non-small cell lung cancer and squamous cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma), appendiceal cancer, small-bowel cancer, esophageal cancer, hepatobiliary cancer (including liver cancer and bile duct carcinoma), bladder cancer, ovarian cancer and a solid tumor, particularly when the cancer or tumor harbors a KRAS G12C mutation.
